SEQUENCE LISTING (21 sequences)

SEQ ID NO: 1 - protein, 14 aa, Homo sapiens
CAGGGSGNTGKLIF

SEQ ID NO: 2 - protein, 13 aa, Homo sapiens
CASSVRSSYEQYF

SEQ ID NO: 3 - protein, 12 aa, Homo sapiens
CAGGGSQGNLIF

SEQ ID NO: 4 - protein, 13 aa, Homo sapiens
CASSIRSSYEQYF

SEQ ID NO: 5 - protein, 12 aa, Homo sapiens
CVVWGSQGNLIF

SEQ ID NO: 6 - protein, 13 aa, Homo sapiens
CASSIRSSYEQYF

SEQ ID NO: 7 - protein, 14 aa, Homo sapiens
CAGGGSSNTGKLIF

SEQ ID NO: 8 - protein, 13 aa, Homo sapiens
CASSIRSSYEQYF

SEQ ID NO: 9 - protein, 17 aa, Homo sapiens
CAMRGSPGGTSYGKLTF

SEQ ID NO: 10 - protein, 13 aa, Homo sapiens
CASSEYSNQPQHF

SEQ ID NO: 11 - protein, 17 aa, Homo sapiens
CAYMTNAGGTSYGKLIF

SEQ ID NO: 12 - protein, 11 aa, Homo sapiens
CASSQGSYGTF

SEQ ID NO: 13 - protein, 15 aa, Homo sapiens
CAGALGGGSQGNLIF

SEQ ID NO: 14 - protein, 13 aa, Homo sapiens
CASSVRSSYEQYF

SEQ ID NO: 15 - protein, 10 aa, Homo sapiens
ELAGIGILTV

SEQ ID NO: 16 - protein, 9 aa, Homo sapiens
NLVPMVATV

SEQ ID NO: 17 - protein, 22 aa, Artificial Sequence
  MP147 for short R1
ACACTCTTTCCCTACACGACGC

SEQ ID NO: 18 - protein, 21 aa, Artificial Sequence
  MP120 for human TRAC
GCAGACAGACTTGTCACTGGA

SEQ ID NO: 19 - DNA, 23 bp, Artificial Sequence
  MP121 for human TRBC
ctctgcttctgatggctcaaaca

SEQ ID NO: 20 - DNA, 55 bp, Artificial Sequence
  MP128 nested R2 plush human TRAC
gtgactggagttcagacgtgtgctcttccgatctgcagggtcagggttctggata

SEQ ID NO: 21 - DNA, 55 bp, Artificial Sequence; identical to SEQ ID NO: 20
  MP129 nested R2 plus human TRBC

(19) United States
(12) Patent Application Publication
(10) Pub. No.: US 2021/0102942 A1
(43) Pub. Date: Apr. 8, 2021

(54) High-throughput method to screen cognate T cell and epitope reactivities in primary human cells

(71) Applicant: Regeneron Pharmaceuticals, Inc., Tarrytown, NY (US)

(72) Inventors: Raquel Deering, Ridgefield, CT (US); Ankur Dhanik, Elmsford, NY (US); Stephane Pourpe, New York, NY (US)

(21) Appl. No.: 17/062,375

(22) Filed: Oct. 2, 2020

Related U.S. Application Data
(60) Provisional application No. 62/910,379, filed on Oct. 3, 2019.

(51) Int. Cl. G01N 33/569

(52) U.S. Cl. CPC G01N 33/56966; G01N 2333/70578

(57) ABSTRACT

Described is an autologous primary immune cell assay in which an individual's own blood cells may be functionally screened against individual antigens, e.g., T cell epitopes, of interest simultaneously without HLA haplotype-specific reagent. Antigen reactivities are linked to individual T cells using an oligonucleotide-tagging hashing tracking system, which is later deconvolved by single cell sequencing.

### CROSS REFERENCE TO RELATED APPLICATIONS

This application claims priority under 35 U.S.C. § 119(e) to U.S. Provisional Patent Application Ser. No. 62/910,379, filed Oct. 3, 2019, the disclosure of which is hereby incorporated by reference in its entirety.

### REFERENCE TO A SEQUENCE LISTING SUBMITTED AS A TEXT FILE VIA EFS WEB

The Sequence Listing written in file 10669_ST25.txt is 5 kilobytes, was created on Oct. 2, 2020, and is hereby incorporated by reference.

### BACKGROUND OF THE INVENTION

As the interest in antigen-specific T cell activity in human disease has come into sharper focus, there is a need to be able to correlate epitope-specific TCR sequences to the cognate epitope presented in the context of HLA. However, identifying which epitopes result in productive T cell activation and the TCR sequences of responding T cells has historically been and continues to be a technically challenging task.

Traditional methods used to assess antigen-specific T cell binding and reactivity include multimer staining and functional T cell assays in which T cells are re-exposed to epitopes to be detected by cytokine or cytolytic responses (e.g. ELISPOT, cell killing assays). While these methods are useful, they can require expensive individual HLA haplotype-specific reagents (multimers) and large blood volumes to assess potential reactivities at a high throughput. Additionally, very few HLA class II (CD4+ T cell) multimers exist so most multimer-based interrogation is focused on HLA class I (CD8+ T cell) reactivities.

Thus, there exists a need for a high throughput method able to provide information that correlates the cognate TCR α and β polypeptides of a TCR with the epitope recognized by the TCR, inter alia.

### SUMMARY OF THE INVENTION

Described herein is an immune cell assay, which may be used on autologous and primary immune cells, in which CD8+ and/or CD4+ T cell responses against multiple T cell epitopes of interest may be simultaneously assayed. Antigen reactivities are linked to individual T cells using a hashtag oligonucleotide (HTO) tracking system, which may be later deconvoluted by single cell sequencing to provide single cell-level information such as: (a) epitope specificity, (b) single cell paired alpha/beta chain TCR sequences, (c) endogenous single cell RNA transcriptome information, (d) cell surface protein expression (e.g., using CITE-seq antibodies), (e) multimer staining (if multimers are included), and any combination thereof. Accordingly, provided herein are the immune cell assay methods, compositions and kits therefor, and uses thereof, e.g., for making a TCR therapeutic.

In one embodiment, a method described herein comprises sorting an activated T cell, e.g., based on its expression of an activation-induced marker (AIM), from a composition comprising other cells, e.g., autologous antigen-presenting cells (APCs), wherein the activated T cell is labeled with an HTO conjugated molecule.

In some embodiments, a method described herein (e.g., for identifying an antigen capable of activating a T cell, and optionally a T cell receptor (TCR) α chain sequence and/or a TCR β chain sequence of a TCR that specifically binds the antigen) comprises

(I) sorting an activated T cell, based on the expression of an activation-induced marker (AIM), from a composition comprising a unique biological sample, which unique biological sample comprises: (a) a T cell and a surface bound Major Histocompatibility Complex (MHC), wherein the T cell is capable of recognizing a peptide presented in the context of the surface bound MHC, (b) a unique antigen, (c) a unique hashtag oligonucleotide (HTO) that may be used to specifically identify (and/or specifically identifies) the unique antigen, wherein the unique HTO is conjugated to a molecule that labels the T cell with the unique HTO, and optionally, (d) medium that supports activation of the T cell, and

(II) performing single cell sequencing analysis on the activated T cell sorted in (I) to identify the unique HTO conjugated to the molecule that labeled the activated T cell with the unique HTO, wherein identifying the unique HTO identifies the antigen capable of activating the activated T cell, and optionally wherein the single cell sequencing analysis also identifies (i) one or more genes expressed by the activated T cell, and/or (ii) TCR α and/or β chain sequences of a TCR expressed by the activated T cell.

Some methods described herein further comprise creating a plurality of biological samples, e.g., unique biological samples, before the sorting step such that composition sorted comprises a plurality of unique biological samples. Some method embodiments comprise, before sorting, the step of creating a plurality of biological samples by equally distributing a collection of cells comprising T cells and antigen presenting cells (APCs) isolated from a subject into individual samples, wherein each biological sample optionally comprises media and cytokines that support T cell and/or APC viability, activation and/or activity.

Some method embodiments comprise, before sorting, the step of creating a plurality unique biological samples by delivering to each of a plurality of biological samples a unique antigen and/or a unique HTO that may be used to specifically identify (and/or specifically identifies) a unique antigen, wherein the unique HTO is conjugated to a molecule that labels a T cell with the unique HTO, wherein each of the plurality of biological samples comprises a collection of cells comprising T cells and APCs isolated from a subject, wherein after delivery of the unique antigen and/or the unique HTO conjugated to a molecule that labels a T cell with the unique HTO, each of the plurality of biological samples becomes a unique biological sample that comprises (a) a collection of cells comprising T cells and APCs isolated from a subject, (b) a unique antigen, (c) a unique HTO that specifically identifies the unique antigen and is conjugated to a molecule that labels the T cell with the HTO, and optionally (d) medium that supports viability, activity, and/or activation of the T cells and APCs. Optionally, the plurality of unique biological samples may be pooled prior to sorting in the methods described herein such that the composition sorted in (I) comprises a plurality of unique biological samples. Some method embodiments herein comprise, before the sorting step, both the step of creating a plurality of biological samples and creating (e.g., from the plurality of biological samples) a plurality of unique biological samples, and optionally pooling the plurality of unique biological samples to create a composition that may be sorted according to a method described herein.

In some methods described herein, the sorting comprises fluorescence activated cell sorting of activated T cells based on the expression of the AIM, e.g., wherein fluorescence activated cell sorting is based on detection of T cells expressing the AIM with a fluorescently labeled antibody that specifically binds the AIM. Such methods may further comprise incubating a unique biological sample (or composition comprising one or a plurality of unique biological samples) with a fluorescently-labeled ligand (e.g., a fluorescently-labeled antibody) that specifically binds the AIM.

In some embodiments, a method described herein further comprises performing functional and/or phenotypic analysis on the activated T cell analyzed by single cell sequencing. In some embodiments, the functional and/or phenotypic analysis is performed before the single cell sequencing analysis. In some embodiments, the functional and/or phenotypic analysis is performed simultaneous with the single cell sequencing analysis. In some embodiments, the functional and/or phenotypic analysis is performed after the single cell sequencing analysis. In some embodiments, the functional and/or phenotypic analysis is performed prior to, simultaneous with, and/or after the single cell sequencing analysis. In some embodiments, the functional and/or phenotypic analysis comprises flow cytometric analysis. In some embodiments, the functional and/or phenotypic analysis comprises CITE-seq analysis. In some embodiments, the functional and/or phenotypic analysis comprises multimer analysis. In some embodiments, the functional and/or phenotypic analysis comprises any combination of flow cytometric analysis, CITE-seq analysis, and multimer analysis. In some embodiments, the further functional and/or phenotypic analysis measures the protein and/or RNA expression levels of one or more of CD3, CD4, CD8, CD25, CD27, CD28, CD45RA, CD62L, HLA DR, CD137/4-1BB, CD69, CD278, CD274, CD279, CD127, CD197, IFNγ, GZMH, GNLY, CD38, CCL3, and LAG3.

In some embodiments, a method described herein comprises identifying a TCR α chain sequence and/or a TCR β chain sequence of a TCR that specifically binds an antigen, preferably wherein the TCR α chain sequence and/or a TCR β chain sequence are a TCR α chain variable region sequence (Vα/Jα sequence) and/or a TCR β chain variable region sequence (Vβ/Jβ sequence), respectively. In some embodiments, a method comprises identifying a TCR α chain sequence and/or a TCR β chain sequence of a TCR that specifically binds the antigen and the method further comprises utilizing the TCR α chain sequence and/or the TCR β chain sequence in making a therapeutic, e.g., a human therapeutic. Alternatively, the method may also comprise identifying TCRδ/TCRγ sequences, e.g., TCRδ/TCRγ variable region sequences.

Also described herein are compositions, which may be used in the methods described herein. In some embodiments, a composition comprises a unique biological sample that comprises: (a) a T cell and a surface bound Major Histocompatibility Complex (MHC), wherein the T cell is capable of recognizing a peptide presented in the context of the surface bound MHC, (b) an antigen, (c) a hashtag oligonucleotide (HTO) that specifically identifies the antigen, wherein the HTO is conjugated to a molecule that labels the T cell with the HTO, and optionally (d) medium that supports activation of the T cell. In some embodiments, a composition comprises more than one biological sample, e.g., a first and a second biological sample, wherein the first biological sample comprises: (a) a first T cell and a first surface bound MHC, wherein the first T cell is capable of recognizing a peptide presented in the context of the first surface bound MHC, (b) a first antigen, and (c) a first HTO that may be used to specifically identify, and preferably specifically identifies, the first antigen, wherein the first HTO is conjugated to a first molecule that labels the first T cell with the first HTO, wherein the second biological sample comprises (a) a second T cell and a second surface bound MHC, wherein the second T cell is capable of recognizing a peptide presented in the context of the second surface bound MHC, (b) a second antigen, and (c) a second HTO that specifically identifies the second antigen, wherein the second HTO is conjugated to a second molecule that labels the second T cell with the second HTO, wherein (i) the first T cell and second T cell are isolated from the same subject, (ii) the first antigen and the second antigen are not identical, (iii) the first molecule that labels the first T cell with the first HTO and the second molecule that labels the second T cell with the second HTO are identical, and the first HTO and the second HTO are not identical, and optionally wherein either or both the first and second biological samples further comprise(s) a medium that supports activation of the first and second T cell.

Also described herein are kits. In some embodiments, a kit described herein comprises a plurality of unique antigens, and a plurality of unique hashtag oligonucleotides (HTOs), each of which plurality of unique HTOs may be used to specifically identify, and preferably each of which specifically identifies, only one of the plurality of unique antigens. In some kit embodiments, each of the plurality of unique HTOs is conjugated to an identical molecule such that the kit comprises a plurality of unique HTO-conjugated molecules. In some kit embodiments as described herein, each of the plurality of unique antigens comprises unique and overlapping peptide sequences from a single protein, e.g., a pathogenic antigen, a tumor associated antigen, or a transplantation antigen.

In some embodiments herein, a surface bound MHC is a cell membrane bound MHC, e.g., the surface bound MHC is expressed on the surface of a cell, e.g., an antigen presenting cell (APC). In some method, composition, kit, or use embodiments, the APC is a monocyte-derived dendritic cell. In some composition, method, kit, or use embodiments, the APC is a dendritic cell. In some method, composition, kit, or use embodiments, the APC is a monocyte. In some method, composition, kit, or use embodiments, the APC is a macrophage. In some method, composition, kit, or use embodiments, the APC is a B cell. In some method, composition, kit, or use embodiments, the surface bound MHC is expressed on the surface of a population of cells, e.g., a population of APCs, e.g., wherein the population of APCs comprises a monocyte-derived dendritic cell, a dendritic cell, a monocyte, a macrophage, a B cell, and any combination thereof. In some embodiments, the T cell and APC(s) are autologous. In some embodiments, the T cell and APC(s) are each isolated from a human donor. In some embodiments, peripheral blood mononuclear cells (e.g., isolated from a human donor) provide the T cell and surface bound MHC (e.g., MHC expressed on the surface of APC(s).

The methods, compositions, kits and uses described herein may be advantageously performed with low volume samples, e.g., low volume human samples. In some embodiments, a collection of cells comprises as sufficient number of peripheral blood mononuclear cells (PBMCs) isolated from a subject, e.g., a human subject, such that the collection of cells may be equally distributed into a plurality of individual biological samples. In some embodiments, the collection of cells comprises a sufficient number of PBMCs such that the collection of cells may be equally distributed into at least two individual biologicals samples, each comprising at least about 1×10⁵ PBMCs, at least about 5×10⁵ PBMCs, or at least about 1×10⁶ PBMCs, e.g., the collection of cells may be derived from about 1 mL, about 3 mL, about 5 mL, about 10 mL, about 15 mL, about 20 mL, or about 50 mL of whole blood isolated from a subject, e.g., a human subject. In some embodiments, the collection of cells comprises a sufficient number of PBMCs such that the collection of cells may be equally distributed into at least three individual samples, each comprising at least about 1×10⁵ PBMCs, at least about 5×10⁵ PBMCs, or at least about 1×10⁶ PBMCs, e.g., the collection of cells may be derived from about 1 mL, about 3 mL, about 5 mL, about 10 mL, about 15 mL, about 20 mL, or about 50 mL of whole blood isolated from a subject, e.g., a human subject. In some embodiments, the collection of cells comprises a sufficient number of PBMCs such that the collection of cells may be equally distributed into at least five individual samples, each comprising at least about 1×10⁵ PBMCs, at least about 5×10⁵ PBMCs, or at least about 1×10⁶ PBMCs, e.g., the collection of cells may be derived from about 1 mL, about 3 mL, about 5 mL, about 10 mL, about 15 mL, about 20 mL, or about 50 mL of whole blood isolated from a subject, e.g., a human subject. In some embodiments, the collection of cells comprises a sufficient number of PBMCs such that the collection of cells may be equally distributed into at least ten individual samples, each comprising at least about 1×10⁵ PBMCs, at least about 5×10⁵ PBMCs, or at least about 1×10⁶ PBMCs, e.g., the collection of cells may be derived from about 1 mL, about 3 mL, about 5 mL, about 10 mL, about 15 mL, about 20 mL, or about 50 mL of whole blood isolated from a subject, e.g., a human subject. In some embodiments, the collection of cells comprises a sufficient number of PBMCs such that the collection of cells may be equally distributed into at least twenty individual samples, each comprising at least about 1×10⁵ PBMCs, at least about 5×10⁵ PBMCs, or at least about 1×10⁶ PBMCs, e.g., the collection of cells may be derived from about 1 mL, about 3 mL, about 5 mL, about 10 mL, about 15 mL, about 20 mL, or about 50 mL of whole blood isolated from a subject, e.g., a human subject. In some embodiments, the collection of cells comprises a sufficient number of PBMCs such that the collection of cells may be equally distributed into at least thirty individual samples, each comprising at least about 1×10⁵ PBMCs, at least about 5×10⁵ PBMCs, or at least about 1×10⁶ PBMCs, e.g., the collection of cells may be derived from about 1 mL, about 3 mL, about 5 mL, about 10 mL, about 15 mL, about 20 mL, or about 50 mL of whole blood isolated from a subject, e.g., a human subject. In some embodiments, the collection of cells comprises a sufficient number of PBMCs such that the collection of cells may be equally distributed into at least fifty individual samples, each comprising at least about 1×10⁵ PBMCs, at least about 5×10⁵ PBMCs, or at least about 1×10⁶ PBMCs, e.g., the collection of cells may be derived from about 1 mL, about 3 mL, about 5 mL, about 10 mL, about 15 mL, about 20 mL, or about 50 mL of whole blood isolated from a subject, e.g., a human subject. In some embodiments, the collection of cells comprises a sufficient number of T cells and antigen presenting cells (APCs), (e.g., dendritic cells (DCs)) such that the collection of cells may be equally distributed into a plurality of individual biological samples. In some embodiments, the collection of cells comprises a sufficient number of APCs and T cells isolated from a subject, e.g., a human subject, such that the collection of cells may be equally distributed into a plurality of individual biological samples, each comprising APCs and T cells (e.g., DCs and T cells) at an APC:T cell ratio of about 1:1, about 1:5, or about 1:10, e.g., wherein each sample comprises at least about 5×10³, 5×10⁴, or 5×10⁵ DCs and about 5×10³, 1×10⁴, 2.5×10⁴, 5×10⁴, 1×10⁶, 2.5×10⁵, 5×10⁵, 1×10⁶, 2.5×10⁶, or 5×10⁶ T cells, e.g., the collection of cells can be derived from about 5 mL, about 10 mL, about 15 mL, about 20 mL ,or about 50 mL of whole blood isolated from a subject, e.g., a human subject.

An antigen used in a method as described herein, or that is part of a composition or kit as described herein, may (I) be an antigen selected from the group consisting of (i) a bacterial antigen or portion thereof, (ii) a viral antigen or portion thereof, (iii) an allergen or portion thereof, (iv) a tumor associated antigen or a portion thereof, and (v) a combination thereof, and/or (II) comprise (i) an amino acid sequence, (ii) a nucleotide sequence, (iii) lysate, and (iv) a combination thereof.

The methods, compositions, kits and uses described herein each comprise a hashtag oligonucleotide (HTO) conjugated to a molecule, which molecule may be used to label cells (e.g., T cells) with the HTO. In some embodiments, the molecule used to label cells with an HTO may comprise a ligand, e.g., an antibody. In some embodiments, the HTO conjugated ligand, e.g., HTO conjugated antibody, binds a cell surface molecule. In some embodiments, the cell surface molecule is ubiquitously expressed by most cells. In some embodiments, the cell surface molecule is or comprises β2 microglobulin. In some embodiments, the cell surface molecule is or comprises CD298. In some embodiments, the cell surface molecule may be expressed selectively by T cells. In some embodiments, the cell surface molecule is or comprises a T cell surface molecule selected from the group consisting of CD2, CD3, CD4, CD8, and any combination thereof. In some embodiments, the cell surface molecule is or comprises CD2. In some embodiments, the cell surface molecule is or comprises CD3. In some embodiments, the cell surface molecule is or comprises CD4. In some embodiments the cell surface molecule is or comprises CD8. In some embodiments, the molecule used to label cells with an HTO may comprise a lipid, e.g., which preferably incorporates itself into a cell membrane, e.g., a cell membrane of a dividing cell. In some embodiments, an HTO conjugated molecule comprises an HTO conjugated lipid, e.g., a lipid-modified oligonucleotide. In some embodiments, the molecule used to label cells with an HTO is or comprises cholesterol. In some embodiments, an HTO conjugated molecule described herein comprises an HTO conjugated cholesterol, e.g., a cholesterol-modified oligonucleotide.

The methods described herein comprise sorting an activated T cell based on the expression of an activated-induced marker (AIM). Accordingly, some method, composition, kit and use embodiments described herein comprise an agent that is useful in such sorting step. In some embodiments, the agent comprises a fluorescently labeled ligand that specifically binds the AIM, e.g. a fluorescently labeled antibody that specifically binds the AIM. In some method, composition, or kit embodiments herein, the AIM is or comprises any marker that is upregulated by a T cells upon activation of the T cell. In some method, composition, kit or use embodiments herein, the AIM is or comprises an AIM selected from the group consisting of CD137/4-1BB, CD107, IFNγ, PD-1, CD40L, OX40, CD25, CD69, CD28, HLA-DR, CX3CR1, TIM3, LAG3, TIGIT, and any combination thereof. In some method, composition, kit, or use embodiments herein, the AIM is or comprises CD137/4-1BB. In some method, composition, kit, or use embodiments herein the AIM is or comprises CD107. In some method, composition, kit, or use embodiments herein the AIM is or comprises IFNγ. In some method, composition, kit, or use embodiments herein the AIM is or comprises PD-1. In some method, composition, kit, or use embodiments herein the AIM is or comprises CD40L. In some method, composition, kit, or use embodiments herein the AIM is or comprises OX40. In some method, composition, kit, or use embodiments herein the AIM is or comprises CD25. In some method, composition, kit, or use embodiments herein the AIM is or comprises CD69. In some method, composition, kit, or use embodiments herein the AIM is or comprises CD28. In some method, composition, kit, or use embodiments herein the AIM is or comprises HLA-DR. In some method, composition, kit, or use embodiments herein the AIM is or comprises CX3CR1. In some method, composition, kit, or use embodiments herein the AIM is or comprises TIM3. In some method, composition, kit, or use embodiments herein the AIM is or comprises LAG3. In some method, composition, kit, or use embodiments herein the AIM is or comprises TIGIT.

A method as described herein may comprise performing functional and/or phenotypic analysis on the activated T cell analyzed by single cell sequencing. Accordingly, in some embodiments, a method, composition, kit or use as described herein may comprise additional reagents, e.g., an antibody and/or MHC multimers, either or both of which may be useful for flow cytometric analysis and/or CITE-seq analysis.

Some method, composition, kit and use embodiments described herein comprise medium that supports the viability, activation, and/or activity of a T cell (and optionally other cell, e.g., an antigen presenting cell, e.g., a dendritic cell) is present. In some embodiments the medium comprises one or more cytokines. In some embodiments, the medium comprises IL-2. In some embodiments, the medium comprises IL-4. In some embodiments, the medium comprises IL-7. In some embodiments, the medium comprises IL-15. In some embodiments, the medium comprises IL-21. In some embodiments, the medium comprises GM-CSF. In some embodiments, the medium comprises FLT3L. In some embodiments, the medium comprises any combination of IL-2, IL-4, IL-7, IL-15, GM-CSF, and FLT3L. In some embodiments, the medium comprises a cytokine selected from the group consisting of IL-2, IL-7, IL-15, GM-CSF, IL-4, and any combination thereof.

Also described herein is the use of a method, composition, and/or kit as described herein for analyzing a T cell mediated immune response of a patient to a vaccine. In some embodiments, a method, composition, and/or kit as described herein may be used for analyzing a T cell mediated immune response of a patient to an immunotherapy. In some embodiments, a method, composition, and/or kit as described herein may be used for analyzing a T cell mediated immune response in a patient during immunotherapy of the patient. In some embodiments, a method, composition, and/or kit as described herein may be used for analyzing T cell responses of a patient to an autoantigen. In some embodiments, a method, composition, and/or kit as described herein may be used for analyzing T cell responses of a patient to a transplant antigen. In some embodiments, a method, composition, and/or kit as described herein may be used to identify one or more TCR variable region sequences of an activated T cell (e.g., a CDR3 sequence of a TCRα chain and/or a CDR3 sequence of a TCRβ chain). In some embodiments, the one or more TCR variable region sequences so identified may be used to create a human therapeutic, e.g., a T cell comprising the one or more TCR variable region sequences identified using a method, composition, and/or kit as described herein.

### BRIEF DESCRIPTION OF THE DRAWINGS

FIG. 1 provides an illustration (not to scale) of a non-limiting exemplary embodiment of the invention. Briefly, unique biological samples, each comprising cells (e.g., whole peripheral blood mononuclear cells, autologous antigen presenting cells and T cells, etc.), a unique antigen, and a unique HTO that identifies the unique antigen, are pooled. The pool of unique biological samples is enriched for activated T cells, which activated T cells may then be analyzed for functional and phenotypic characteristics. Detailed steps of the method are described herein below.

FIG. 2A provides Fluorescence Activated Cell Sorting (FACS) dot blots of T cell:dendritic cell co-cultures after priming with DMSO, CMV pp65 or MART 1 antigens (a) after pre-expansion and before re-stimulation and (b) after a 24-hour re-stimulation with the same antigens. Cell surface expression of CD8 and functional activation measured by CD137/4-1BB or multimer staining was assessed using fluorescently-tagged monoclonal antibodies (CD8 and CD137/4-1BB) or dextramer multimers by flow cytometry. FIG. 2B provides the percentage of CD8⁺T cells (y-axis) isolated from four different donors (x-axis) that were incubated with DMSO or CMV pp65, and that bound to a negative multimer or a pp65 labeled multimer (top panel) or anti-4-1BB antibody (bottom panel). * Negative multimer=multimer with irrelevant peptide; FMO=fluorescence minus one control

FIG. 3A shows the percentage of functional CD8+ T cells (y-axis) within a population of whole peripheral blood mononuclear cells (PBMCs) from healthy HLA-A*0201+human donors (HD3 and HD27; x-axis) after a 10-day pre-expansion and a 24-hour re-stimulation with either DMSO (Baseline) or MART1 (ELAGIGILTV: SEQ ID NO:15) synthetic short peptides (MART-1 re-exposure). Multimers were used to stain the baseline population and anti-CD137/4-1BB antibodies were used to stain the re-stimulated population. * FMO=fluorescence minus one control; Negative multimer=multimer with irrelevant peptide. FIG. 3B provides violin plots to show the clonal frequency as a percentage of T cells (top panel; y-axis) and clonal size as the number of T cells (bottom panel; y-axis) of all TCR clones specific to MARTI from donor HD27 and identified by CD137/4-1BB or multimer staining (x-axis). Embedded in the violin plots are box plots showing the median, upper, and lower quartiles and the interquartile range (distance between the upper and lower quartiles).

FIGS. 4A-B provide data derived from unique biological samples comprising T cells pre-expanded and re-stimulated with hCMV pp65 peptide and incubated with one of the following unique antigens: EBV YVL-9, hCMV pp65, EBV LMP2A, EBV BMLF1, or Influenza M virus. FIG. 4A shows data from an ELISPOT assay in which the number of peptide-specific T cells were enumerated by measuring IFNγ production (SFC/2×10⁶; y-axis) by these biological samples. SPC=spot forming colonies. FIG. 4B shows dot plots from flow cytometric analysis of these biological samples stained with anti-CD137/4-1BB (y-axis) and anti-CD8 (x-axis) antibodies. The percent CD137/4-1BB+CD8+ cells incubated with DMSO was 0.25%, with EBV YVL-9 peptide was 1.27%, with CMV pp65 peptide was about 26.2%, with EBV LMP2A peptide was about 3.21%, with EBV BMLF1 peptide was about 9.67%, or with Influenza virus peptide was about 5.2%.

FIG. 5 provides a non-limiting illustration (not-to scale) of a non-limiting embodiment of the invention whereby unique biological samples comprising PBMCs, a unique antigen polypeptide, e.g., as described in FIG. 4, and a unique hashtag oligonucleotide conjugated anti-CD2 antibody (HTO; 1-6) are pooled, enriched for those cells expressing CD137/4-1BB and CD8, and analyzed by single cell sequencing (5′ scSEQ) analysis. Although 6 hashed wells are shown, e.g., for each of the unique biological samples stimulated with DMSO, EBV YVL-9, hCMV pp65, EBV LMP2A, EBV BMLF1, or Influenza M virus, the DMSO population would not ultimately provide many CD8⁺CD137/4-1BB⁺ T cells for single cell sequencing analysis.

FIG. 6A shows the level (on a scale of 0 to 2.5) of hashtag oligonucleotides (HTO-1, HTO-2, HTO-3, HTO-4, HTO-5) (y-axis) associated with individual CD137/4-1BB⁺CD8⁺ T cells enriched from unique biological samples that were each stimulated with a unique antigen (EBV YVL-9, CMV pp65, EBV LMP2A, EBV BMLF1, or Influenza M; y-axis). Also shown are those CD137/4-1BB⁺CD8⁺ T cells for which more than one HTO was sequenced (doublets) or for which no HTO was sequenced (No HTO). FIG. 6B provides a schematic (not to scale) example of how FIG. 6A would look if each unique antigen, e.g., each of EBV YVL-9, CMV pp65, EBV LMP2A, EBV BMLF1, or Influenza M, equally expanded its associated unique biological sample. FIG. 6C shows the number of hashed cells from the sorted pool (y-axis) of FIG. 6A for each population of CD137/4-1BB⁺CD8⁺ T cells identified for reactivity against EBV YVL-9, CMV pp65, EBV LMP2A, EBV BMLF1, Influenza M and corresponding to HTO-1, HTO-2, HTO-3, HTO-4, and HTO-5, respectively. Also shown are the number CD137/4-1BB⁺CD8⁺ T cells for which more than one HTO was sequenced (doublets) or for which no HTO was sequenced (No HTO). The top panel of FIG. 6D provides graphs that show the normalized dextramer expression (counts; y-axis) by enriched CD137/4-1BB⁺ T cells hashed with HTO 40 (which, like HTO-5, identifies the Influenza M antigen), HTO 47 (which, like HTO-4, identifies the EBV BMLF1 antigen) or HTO-48 (which, like HTO-2, identifies the CMV pp65 antigen) after staining with dextramers loaded with various peptides (x-axis). The bottom panel of FIG. 6D provides the clone size of T-cell clones that were hashtagged with HTO-40, HTO-47, and HTO-48 and enriched for CD137/4-1BB expression (x-axis) and the clone size of those clones identified in the experiment depicted in the top panel. The total number of unique clones, e.g., the total clones, is denoted by TC. The number of clones that show high expression of dextramer corresponding to the hashed antigen, e.g., the overlapping clones, is denoted by OC.

FIG. 7A shows the seven unique clusters (clusters 0-6) resolved from RNA transcriptome analysis of individual CD137/4-1BB⁺CD8⁺ T cells enriched from unique biological samples that were each stimulated with a unique antigen (EBV YVL-9, CMV pp65, EBV LMP2A, EBV BMLF1, or Influenza M; y-axis). Each dot represents a single cell. FIG. 7B provides a TCR clonality maps that shows the individual T cells corresponding to cognate reactivities against EBV YVL-9, CMV pp65, EBV LMP2A, EBV BMLF1, or Influenza M and the relative TCR clone size for each reactivity. Each dot represents a single cell.

FIG. 8A provides the relative protein expression level of phenotypic and functional T cell markers (CD3, CD4, CD8a, CD45RA, CD62L, HLA-DR, CD274-PDL1, CD279-PD1, CD127, CD25, CD27, CD28, CD137/4-1BB, CD69, CD278/ICOS, CD197/CCR7) by individual CD137/4-1BB⁺ T cells enriched from unique biological samples that were each stimulated with a unique antigen (EBV YVL-9, CMV pp65, EBV LMP2A, EBV BMLF1, or Influenza M; y-axis), where an individual cell is represented by a dot and clustered according to the RNA transcriptome analysis depicted in FIG. 7. The corresponding RNAseq transcript expression levels of these same phenotypic and functional T cell markers by each cell, each of which is represented by a dot, are shown in FIG. 8B

FIG. 9A shows the 5 unique clusters based on HTO identification (1-5) of individual CD137/4-1BB+ T cells enriched from unique biological samples that were stimulated with a unique HPV antigen identified by HTO-1, HTO-2, HTO-3, HTO-4, or HTO-5. Each cluster is identified both by number and grayscale gradation. Cluster “M” are those individual cells that identified with multiple HTO. Each dot represents an individual cell. FIG. 9B replicates the cluster map of FIG. 9A and shows cells that represent TCR clones that are not shared across HTO clusters, e.g., expresses a TCR specific for the unique antigen identified by the HTO. Cells of the same clone are represented by the same grayscale coloration. FIG. 9C provides replicates of the cluster maps of FIGS. 9A-B to show the relative RNA expression levels of CD137/4-1BB, IFNγ, GZMH, GNLY, CD38, CCL3, and LAG3 by individual cells. Also shown is the CD137/4-1BB protein expression level (CITE-Seq) by these cells.

### DESCRIPTION

Oligonucleotide (oligo)-tagged antibodies were developed as a way to bypass traditional flow cytometric analysis. See, e.g., WO2018144813, incorporated herein in its entirety by reference. Such oligo-tagged antibodies may be used as a tool to bind proteins on the surface of live cells to aid in single cell tracking for single cell RNA sequencing (scRNA SEQ) experiments. A method described in Stoeckius (2017) bioRxiv (also printed in (2018) Genome Biology 19: 224) uses oligo-tagged antibodies with a single protein specificity that is expressed on all target cells as well as unique oligonucleotide tags (also referred to as hashtag oligonucleotides or HTOs) having unique sequences per sample to track individual samples that are ultimately pooled for sequencing library preparation. In this method, each cell may be tagged with a unique oligonucleotide sequence that identifies the sample from whence the cell came. This oligonucleotide sequence is detected and included in the sequencing library, so the identity of the sample can be determined from the resulting sequencing information. Traditionally, hashing antibodies are used to pool multiple samples, e.g., multiplex the samples, into one single cell sequencing (scSEQ) library preparation to normalize data and improve efficiency.

Use of HTOs in functional assays, e.g., for the characterization specific T cell responses has been previously described, wherein the HTO is conjugated to a multimer of Major Histocompatibility Complex (MHC), see, e.g., Bentzen et al. (2016) Nature Biotechnology 34:1037-45. MHC are expressed by antigen presenting cells (APCs) and present peptides to T cells that recognize the peptides. Dogmatically, CD8⁺ T cells pair with MHC I while CD4⁺ T cells pair with MHC II. Also, the extreme polymorphism of MHC makes it important to know which alleles are recognized by the T cells as self, such that any response can be said to be due to the presentation of the peptide itself, not the presentation of a foreign MHC. Thus, to be able to effectively stimulate antigen-specific T cells and characterize peptide specific T cell responses, peptide must be presented in an MHC that matches in class and haplotype to the corresponding T cell.

Previously, peptide specific T cell responses were characterized by functional assays such as proliferation assays, chromium-based cytotoxicity assays, Ca²⁺ flux assays, and more commonly, cytokine detection assays such as ELISPOT and intracellular cytokine flow cytometry staining. Klinger et al. (2015) PLoS One DOI:10.1371/journal.pone.0141561, which describes multiplexing of such assays. However, these functional assays were limited in that they could neither delineate the antigen specificity nor characterize the response at a single cell level. Some of these limitations were overcome by flow cytometric MHC tetramer staining. With flow cytometric MHC tetramer staining, specific T cell responses could be evaluated with fluorophore conjugated MHC multimers loaded with a peptide of interest. Identification of T cells that specifically bind, and likely are activated with, the MHC multimer loaded with a peptide of interest was achieved by sorting for those cells that were bound to the fluorescent MHC multimer, and sometimes other antibodies. The transition from fluorescently labeled MHC multimers to HTO conjugated MHC multimers removes the limiting factor of the small number of fluorescent tags available to characterize the activated T cells. Additionally, similar to hashing antibodies, hashing MHC multimers are useful to track individual samples that are ultimately pooled for sequence analysis, whereby the HTO sequence is detected and included in the sequencing library, and identifies the MHC/peptide combination that bound to the T cell being analyzed. However, unlike the methods described in Stoeckius (2017) bioRxiv (also printed in (2018) Genome Biology 19: 224), supra, use of an HTO conjugated MHC multimer provides for more than the tracking of a sample in that such use also provides a functional analysis, e.g., the identification of an MHC/peptide combination that is able to bind a specific T cell.

Described herein is a functional assay for tracking antigen-specific T cell responses at the single cell level, but which functional assay does not require (although it also does not prohibit) the use MHC multimers. Generally, the methods described herein use hashing molecules to track activated T cells from individual assay wells. Only after cells from all wells are uniquely labeled with one or more oligo-tagged molecules, e.g., that may be incorporated into a cell membrane (e.g., one or more oligo-tagged lipids) and/or that bind to one or more ubiquitous cell surface markers (e.g., one or more oligo-tagged antigen-biding proteins), respectively, are different unique cultures pooled. Since the cells are hashtagged, their source and cognate antigen may be determined and there is no need to keep samples separate. After pooling, the functional assay of flow cytometric analysis for an activation-induced marker (AIM) may be used to sort those cells which were activated from those cells in the pool that were not activated.

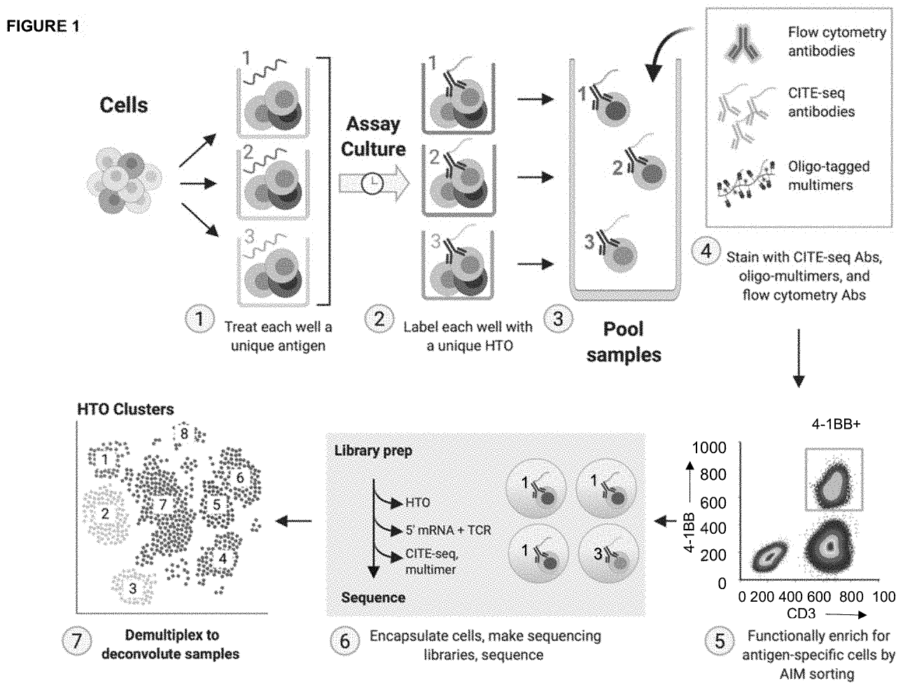

A non-limiting exemplary illustration of the method described herein is illustratively depicted in FIG. 1. As shown in this non-limiting example, in step (1) a unique antigen, e.g., a unique T cell epitope (e.g., “1”, “2”, “3”), which may be protein, peptide, RNA, cell, cell lysate, etc. and/or a single stimuli or pools of stimuli, is added to each of individual wells comprising one of a plurality of biological samples, wherein each of the plurality of biological samples comprises a T cell and an MHC that is recognized by the T cell, e.g., wherein each of the plurality of samples comprises autologous peripheral blood mononuclear cells (PBMCs). The biological samples are cultured with the unique antigen for a time sufficient for the upregulation of an activation-induced marker by activated T cells (e.g., 6-72 hours). In some non-limiting exemplary embodiments, only an overnight culture of the biological sample with antigen (e.g., from about 18-24 hours) is needed for upregulation of the AIM by activated T cells, e.g., during re-stimulation of the activated T cells. In some non-limiting embodiments, the biological samples may first be primed with the antigen, e.g., for about one or two weeks, e.g., for about 7-14 days, to allow pre-expansion of reactive T cells, before the overnight re-stimulation culture. Following re-stimulation, in step (2), each individual well is incubated with a unique hashtag oligonucleotide (HTO) conjugated to a molecule (e.g., a lipid, an antibody, etc.) that incorporates into the cell membrane and/or specifically binds a cell surface marker expressed by the T cell regardless of activation state (e.g., β2 microglobulin, CD2, CD298, CD3, CD4, and/or CD8, etc.), where each unique HTO (e.g., “1”, “2”, “3”) identifies the unique antigen in each the individual well. In step (3), all unique biological samples are multiplexed, e.g., pooled, and after pooling, in step (4), the composition comprising the pool of unique biological samples is incubated with an agent useful for detecting an activation-induced marker expressed by activated T cells (e.g., CD137/4-1BB) and other single cell sequencing and flow cytometry reagents as desired, such as including but not limited to CITE-seq antibodies, fluorescently tagged antibodies, and oligonucleotide-tagged multimers. Although the non-limiting embodiment depicted in FIG. 1 illustrates addition of these additional reagents after pooling, in other non-limiting embodiments, these additional reagents may be added prior to pooling. In step (5), cells labeled with the agent useful for detecting the activation induced marker, e.g., a fluorescently labeled antibody that specifically binds the activation induced marker, and another T cell marker (e.g., CD137/4-1BB+ CD3+ T cells) are functionally enriched by AIM fluorescence activated cell sorting (FACS). In step (6) the transcriptome of each of the enriched cells is then analyzed, e.g., the population of sorted cells is encapsulated into 10× Genomics single cell Gel Bead-In Emulsions (GEMS) to partition the cells into single cells, and RNA sequencing is performed on each cell. In the non-limiting embodiment shown in FIG. 1, 5′ sequencing libraries for the HTO, transcriptome (5′mRNA), TCR-seq, CITE-seq, and/or oligo-multimers are generated for high throughput single cell sequencing in step (6). In step (7) individual HTO clusters are bioinformatically demultiplexed to elucidate to which antigen individual cells were exposed.

The functional assay described provides many benefits. For example, the methods described herein may be fully personalized, e.g., by the use of autologous T cells and MHC. Additionally, it allows for the interrogation of many reactivities simultaneously, even if the biological sample to be tested is limited. Cognate antigen/T-cell reactivity may be identified for a single cell or at a pooled cell level. Use of reagents that are not MHC-specific allows for flexible application of the method across patient samples, as well as the ability to capture information in a non-MHC restricted manner, e.g., both CD4+ and CD8+ T cell information may be captured simultaneously, and use of a functional phenotype (e.g., an activation-induced marker) helps identify and evaluate only activated T cells. Additionally, the method described herein is compatible with follow-on methods of evaluating the phenotype and transcriptome of activated T cells in a fast and cost-effective manner that may inform personalized therapy development and/or decisions. In this way, immune responses to therapy (vaccines, immunotherapy, etc.), e.g., T cell reactivity to vaccine-encoded antigens, viral antigens, and/or tumor antigens may be assessed. Similarly, immune monitoring of autoimmune reactivities, e.g., T cell reactivities to self-antigens, may also be assayed. The methods described herein may be useful for TCR discovery and therapeutic development, e.g., to screen for TCRs of interest across a number of antigens of interest and/or for TCR:epitope binding discovery and algorithm generation. For example, compilation of the epitope:TCR sequence data provided by the methods described herein may aid in the discovery of haplotype-specific rules regarding the TCR sequence(s) and/or structural features associated with specific HLA-peptide binding.

Accordingly, described herein are methods comprising one or more of the following steps:

hashing a biological sample comprising a T cell and an MHC, e.g., incubating the biological sample with a unique antigen (e.g., a T cell epitope) and a unique barcode (e.g., hashtag oligonucleotide) to form a unique biological sample,

pooling a plurality of unique biological samples,

enriching for activated T cells based on a functional assay (e.g., AIM sorting, e.g., with a fluorescently labeled antibody to an activation induced marker, e.g., CD137/4-1BB, CD107, IFNγ, PD-1, CD40L, OX40, CD25, CD69, CD28, HLA-DR, CX3CR1, TIM3, LAG3, and/or TIGIT),

performing sequencing methods, and optionally other well-known methods (e.g., CITE-seq analysis, flow cytometric analysis, and/or multimer staining) on the activated cells, e.g., on a single cell basis, to identify (a) the unique barcode of the activated T cell and thereby the antigen that activated the T cell and optionally (b) other sequences that may be useful to identify, e.g., the TCR α and β sequences of the activated T cell, e.g., for therapeutic development.

Definitions

Unless defined otherwise, all technical and scientific terms used herein have the same meaning as commonly understood by one of ordinary skill in the art.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a meaningful range of a value. The allowable variation encompassed by the term “about” or “approximately” depends on the particular system under study, and can be readily appreciated by one of ordinary skill in the art.

T cells bind epitopes on small antigenic determinants on the surface of antigen-presenting cells that are associated with a major histocompatibility complex (MHC). T cells bind these epitopes through a T cell receptor (TCR) complex on the surface of the T cell. T cell receptors are heterodimeric structures composed of two types of chains: an α (alpha) and β (beta) chain, or a γ (gamma) and δ (delta) chain. The α chain is encoded by the nucleic acid sequence located within the α locus on human chromosome 14, which also encompasses the entire δ locus, and the β chain is encoded by the nucleic acid sequence located within the β locus on human chromosome 7. The majority of T cells have an αβ TCR; while a minority of T cells bear a γδ TCR. Although the α and β chains are commonly referred to herein, the methods, compositions and kits described herein may be similarly applied to γδ TCR chains.

T-cell receptor α and β polypeptides (and similarly γ and δ polypeptides) are linked to each other via a disulfide bond. Each of the two polypeptides that make up the TCR contains an extracellular domain comprising constant and variable regions, a transmembrane domain, and a cytoplasmic tail (the transmembrane domain and the cytoplasmic tail also being a part of the constant region). The variable region of the TCR determines its antigen specificity, and similar to immunoglobulins, comprises 3 complementary determining regions (CDRs), e.g., CDR1, CDR2, and CDR3. Also similar to immunoglobulin genes, T cell receptor variable gene loci (e.g., TCRα and TCRβ loci) contain a number of unrearranged V(D)J segments (variable (V), joining (J), and in TCRβ and δ, diversity (D) segments). During T cell development in the thymus, TCRα variable gene locus undergoes rearrangement, such that the resultant TCR α variable domain is encoded by a specific combination of VJ segments (Vα/Jα sequence); and TCRβ variable gene locus undergoes rearrangement, such that the resultant TCR β variable domain is encoded by a specific combination of VDJ segments (Vβ/Dβ/Jβ sequence). The TCR α and β variable domains, in particular the CDR1, CDR2, and CDR3 and more particularly the CDR3, provide the specificity with which the TCR binds an MHC.

The terms “major histocompatibility complex,” and “MHC” encompass the terms “human leukocyte antigen” or “HLA” (the latter two of which are generally reserved for human MHC), naturally occurring MHC, individual chains of MHC (e.g., MHC class I α (heavy) chain, β2 microglobulin, MHC class II α chain, and MHC class II β chain), individual subunits of such chains of MHC (e.g., α1, α2, and/or α3 subunits of MHC class I α chain, α1-α2 subunits of MHC class II α chain, β1-β2 subunits of MHC class II β chain) as well as portions (e.g., the peptide-binding portions, e.g., the peptide-binding grooves), mutants and various derivatives thereof (including fusions proteins), wherein such portion, mutants and derivatives retain the ability to display an antigenic peptide for recognition by a T cell receptor (TCR), e.g., an antigen-specific TCR. An MHC I comprises a peptide binding groove formed by the α1 and α2 domains of the heavy α chain that can stow a peptide of around 8-10 amino acids. Despite the fact that both classes of MHC bind a core of about 9 amino acids (e.g., 5 to 17 amino acids) within peptides, the open-ended nature of the MHC class II peptide binding groove (the α1 domain of a class II MHC α polypeptide in association with the β1 domain of a class II MHC β polypeptide) allows for a wider range of peptide lengths. Peptides binding MHC class II usually vary between 13 and 17 amino acids in length, though shorter or longer lengths are not uncommon. As a result, peptides may shift within the MHC class II peptide binding groove, changing which 9-mer sits directly within the groove at any given time.

The term “antigen” encompasses any agent (e.g., protein, peptide, polysaccharide, glycoprotein, glycolipid, nucleotide, portions thereof, or combinations thereof) that, when introduced into an immunocompetent host is recognized by the immune system of the host and elicits an immune response by the host. The T-cell receptor (TCR) recognizes a peptide presented in the context of a major histocompatibility complex (MHC) as part of an immunological synapse. The peptide-MHC (pMHC) complex is recognized by TCR, with the peptide (antigenic determinant) and the TCR idiotype providing the specificity of the interaction. Accordingly, the term “antigen” encompasses peptides presented in the context of MHCs, e.g., peptide-MHC complexes, e.g., pMHC complexes. The peptide displayed on MHC may also be referred to as an “epitope” or an “antigenic determinant”. The terms “peptide,” “antigenic determinant,” “epitopes,” etc., encompass not only those presented naturally by antigen-presenting cells (APCs), but may be any desired peptide so long as it is recognized by a T cell when presented appropriately to the T cell. For example, a peptide having an artificially prepared amino acid sequence may also be used as the epitope.

TCR engagement with cognate pMHC is generally short-lived although this interaction may be stabilized by the “avidity effect” afforded by incorporating multiple pMHC on a single backbone, e.g., surface, e.g., the use of multimers, e.g., tetramers, dextramers, etc. Various pMHC multimerization platforms have been utilized, many of which are commercially available. See, e.g., Wooldridge et al. (2009) Immunol. 126:147-64. In order to accommodate such avidity effect, in some embodiments, MHC herein is preferably surface bound such that an appropriate density of MHC may be achieved.

Non-limiting exemplary surfaces to which MHC may be bound in non-limiting embodiments disclosed herein include
a. cell membranes, e.g., wherein the MHC is expressed on the surface of an antigen presenting cell (e.g., a professional antigen presenting cell such as a dendritic cell, monocyte, macrophage, and B cell), the surface of a liposome, the envelope membrane of a viral vector, etc.,
b. a bead,
c. a cell culture dish, e.g., a well of a multi-well plate, and
d. as a multimer, e.g., a tetramer, dextramer, etc.

An antigen may comprise synthetic peptides, protein, mRNA, viruses, viral vectors, DNA, live cells, cell lysates, etc. In some non-limiting embodiments, an antigen is a tumor associated antigen, including peptide portions thereof. In such an embodiment, the tumor associated antigen may be selected from the group consisting of ALK, BAGE proteins, BIRC5 (survivin), BIRC7, CA9, CALR, CCR5, CD19, CD20 (MS4A1), CD22, CD27, CD30, CD33, CD38, CD40, CD44, CD52, CD56, CD79, CDK4, CEACAM3, CEACAM5, CLEC12A, EGFR, EGFR variant III, ERBB2 (HER2), ERBB3, ERBB4, EPCAM, EPHA2, EPHA3, FCRL5, FLT3, FOLR1, GAGE proteins, GD2, GD3, GPNMB, GM3, GPR112, IL3RA, KIT, KRAS, LGR5, EBV-derived LMP2, L1CAM, MAGE proteins, MLANA, MSLN, MUC1, MUC2, MUC3, MUC4, MUC5, MUC16, MUM1, ANKRD30A, NY-ESO1 (CTAG1B), OX40, PAP, PAX3, PAX5, PLAC1, PRLR, PMEL, PRAME, PSMA (FOLH1), RAGE proteins, RET, RGS5, ROR1, SART1, SART3, SLAMF7, SLC39A6 (LIV1), STEAP1, STEAP2, TERT, TMPRSS2, Thompson-nouvelle antigen, TNFRSF17, TYR, UPK3A, VTCN1, WT1.

In another embodiment, an antigen may be associated with an infectious disease. In such embodiments, for example, a biological sample may become a unique biological sample with the addition of an infectious agent or epitope derived therefrom. In one such embodiment, the infectious disease associated antigen may be a viral antigen and the viral antigen is selected from the group consisting of HIV, hepatitis A, hepatitis B, hepatitis C, herpes virus (e.g., HSV-1, HSV-2, CMV, HAV-6, VZV, Epstein Barr virus), adenovirus, influenza virus, flavivirus, echovirus, rhinovirus, coxsackie virus, coronavirus (e.g., SARS-CoV-2), respiratory syncytial virus, mumps virus, rotavirus, measles virus, rubella virus, parvovirus, vaccinia virus, HTLV, dengue virus, papillomavirus, molluscum virus, poliovirus, rabies virus, JC virus, ebola virus, and arboviral encephalitis virus antigen. In another such embodiment, the infectious disease associated antigen may be a bacterial antigen and the bacterial antigen is selected from the group consisting of chlamydia, rickettsia, mycobacteria, staphylococci, streptococci, pneumococci, meningococci, gonococci, klebsiella, proteus, serratia, pseudomonas, legionella, diphtheria, salmonella, bacilli, cholera, tetanus, botulism, anthrax, plague, leptospira, and Lyme disease bacterial antigen.

The term “biological sample” as used in the methods described herein refers to a culture comprising a biologically active cell, an activator of the biologically active cell, and optionally, medium that supports the viability of the cell and/or the biological activation, e.g., activity, of the cell. The biologically active cell may be a homogenous population of cells, such as isolated cells of a particular type (e.g., T cells), or a mixture of different cell types (e.g., peripheral blood mononuclear cells (PBMCs), a co-culture of antigen presenting cells (APCs) and T cells, a co-culture of dendritic cells (DCs) and T cells, etc.), which may be isolated from or comprise a biological fluid or tissue isolated from a subject, e.g., a human or mammalian or other species subject. Biological fluid or tissue may include, as a non-limiting example, serum, plasma, whole blood, peripheral blood, saliva, urine, vaginal or cervical secretions, amniotic fluid, placental fluid, cerebrospinal fluid, serous fluids, or mucosal secretions (e.g., buccal, vaginal or rectal). Still other samples include a blood-derived or biopsy-derived biological sample or tissue, e.g., tissues comprising tumor infiltrating lymphocytes (e.g., tumors), indurations, etc.

Some non-limiting biological samples disclosed herein comprise a T cell and a surface-bound MHC presenting an antigen (e.g., a T cell epitope), e.g., the biologically active cell is a T cell and the activator is a surface bound MHC presenting an antigen (e.g., a T cell epitope). Some non-limiting biological samples disclosed herein comprise a T cell, a surface-bound MHC presenting an antigen (e.g., a T cell epitope), and one or more cytokines that support the viability, activation, and/or activity of the T cell, e.g., the biologically active cell is a T cell, the activator is a surface bound MHC presenting an antigen (e.g., a T cell epitope) and the medium comprises one or more cytokine that supports the viability, activation, and/or activity of the T cell. In some embodiments, a cytokine that supports the viability, activation, and/or activity of the T cell comprises an interleukin selected from the group consisting of IL-2, IL-4, IL-7, IL-15, IL-21 and a combination thereof. Some non-limiting biological samples disclosed herein comprise a T cell and a surface-bound MHC presenting an antigen, wherein the MHC is expressed on the surface of an antigen presenting cell, e.g., a somatic cell, which may optionally be a professional antigen presenting cell selected from the group consisting of a monocyte derived dendritic cell, a dendritic cell, a monocyte, a macrophage, and a B cell. These non-limiting biological samples comprising a T cell and a surface-bound MHC presenting an antigen, wherein the MHC is expressed on the surface of an antigen presenting cell may optionally further comprise a cytokine that supports the viability, activation, and/or activity of the T cell (e.g., IL-2, IL-4, IL-7, IL-15, and/or IL-21) and/or a cytokine that supports the viability, activation, and/or activity of the antigen presenting cell (e.g., GM-CSF, FLT3L, and/or IL-4). Additional cytokines or combinations of cytokines useful for supporting the viability, activation, and/or activity of a T cell and/or antigen presenting cell (and the amounts of the same for supporting the viability, activation, and/or activity of a T cell and/or antigen presenting cell) are well-known in the art. In some embodiments, additional factors that activate APCs are included, e.g., IFNα, LPS, poly-IC, TNF, IL-1β, IL-6, PGE2, etc. in the medium that supports the viability of the cell.

A biological sample is often obtained from, or derived from a specific source, subject or patient

An “individual” or “subject” or “animal” refers to humans, veterinary animals (e.g., cats, dogs, cows, horses, sheep, pigs, etc.) and experimental animal models of diseases (e.g., mice, rats). In one embodiment, the subject is a human.

In a non-limiting embodiment herein, a biological sample comprises peripheral blood mononuclear cells (PBMCs) derived from a subject. The biological samples described herein may comprise newly isolated PBMCs, freshly thawed PBMCs that have been cryopreserved, or PBMCs that have been primed, e.g., cultured for about a week in the presence of antigen to expand memory reactivities and increase assay signal.

Generally, a biological sample (e.g., a unique biological sample) as described herein comprises T cells and surface bound MHC in sufficient numbers to support activation of the T cells in response to an antigen, e.g., at least 1×10⁵, 5×10⁵, 1×10⁶, or more whole peripheral blood mononuclear cells. The combination of hashing and multiplexing advantageously provides for the methods described herein to be performed on low blood volume samples, e.g., low volume human blood samples, since 1 mL whole (human) blood may comprise anywhere from 5×10⁵ to 3×10⁶ peripheral blood mononuclear cells (PBMCs) and/or may be used to isolate anywhere from 5×10³ to 5×10⁵ APCs (e.g., dendritic cells) and anywhere from 5×10³ to 5×10⁶ T cells. As a non-limiting example, a collection of cells derived from 10 mL of whole blood isolated from a subject may comprise anywhere from 5×10⁶ to 3×10⁷ PBMCs such that the collection of cells may be equally distributed into a plurality of individual biological samples, e.g., at least 20 biological samples each comprising about 1×10⁵ to 1×10⁶ PBMCs and/or 1×10⁵ to 5×10⁵ DCs and 1×10⁵ to 5×10⁶ T cells, etc., which may then be pooled (after addition of a unique antigen and/or unique HTO to each) and assayed according to a method described herein. Accordingly, in some embodiments, a collection of cells comprises a sufficient number of peripheral blood mononuclear cells (PBMCs) such that the collection of cells may be equally distributed into a plurality of biological samples. In some embodiments, the collection of cells comprises a sufficient number of PBMCs such that the collection of cells may be equally distributed into at least two individual biologicals samples, each comprising at least about 1×10⁵ PBMCs, at least about 5×10⁵ PBMCs, or at least about 1×10⁶ PBMCs, e.g., the collection of cells may be derived from about 1 mL, about 3 mL, about 5 mL, about 10 mL, about 15 mL, about 20 mL, or about 50 mL of whole blood isolated from a subject, e.g., a human subject. In some embodiments, the collection of cells comprises a sufficient number of PBMCs such that the collection of cells may be equally distributed into at least three individual samples, each comprising at least about 1×10⁵ PBMCs, at least about 5×10⁵ PBMCs, or at least about 1×10⁶ PBMCs, e.g., the collection of cells may be derived from about 1 mL, about 3 mL, about 5 mL, about 10 mL, about 15 mL, about 20 mL, or about 50 mL of whole blood isolated from a subject, e.g., a human subject. In some embodiments, the collection of cells comprises a sufficient number of PBMCs such that the collection of cells may be equally distributed into at least five individual samples, each comprising at least about 1×10⁵ PBMCs, at least about 5×10⁵ PBMCs, or at least about 1×10⁶ PBMCs, e.g., the collection of cells may be derived from about 1 mL, about 3 mL, about 5 mL, about 10 mL, about 15 mL, about 20 mL, or about 50 mL of whole blood isolated from a subject, e.g., a human subject. In some embodiments, the collection of cells comprises a sufficient number of PBMCs such that the collection of cells may be equally distributed into at least ten individual samples, each comprising at least about 1×10⁵ PBMCs, at least about 5×10⁵ PBMCs, or at least about 1×10⁶ PBMCs, e.g., the collection of cells may be derived from about 1 mL, about 3 mL, about 5 mL, about 10 mL, about 15 mL, about 20 mL, or about 50 mL of whole blood isolated from a subject, e.g., a human subject. In some embodiments, the collection of cells comprises a sufficient number of PBMCs such that the collection of cells may be equally distributed into at least twenty individual samples, each comprising at least about 1×10⁵ PBMCs, at least about 5×10⁵ PBMCs, or at least about 1×10⁶ PBMCs, e.g., the collection of cells may be derived from about 1 mL, about 3 mL, about 5 mL, about 10 mL, about 15 mL, about 20 mL, or about 50 mL of whole blood isolated from a subject, e.g., a human subject. In some embodiments, the collection of cells comprises a sufficient number of PBMCs such that the collection of cells may be equally distributed into at least thirty individual samples, each comprising at least about 1×10⁵ PBMCs, at least about 5×10⁵ PBMCs, or at least about 1×10⁶ PBMCs, e.g., the collection of cells may be derived from about 1 mL, about 3 mL, about 5 mL, about 10 mL, about 15 mL, about 20 mL, or about 50 mL of whole blood isolated from a subject, e.g., a human subject. In some embodiments, the collection of cells comprises a sufficient number of PBMCs such that the collection of cells may be equally distributed into at least fifty individual samples, each comprising at least about 1×10⁵ PBMCs, at least about 5×10⁵ PBMCs, or at least about 1×10⁶ PBMCs, e.g., the collection of cells may be derived from about 1 mL, about 3 mL, about 5 mL, about 10 mL, about 15 mL, about 20 mL, or about 50 mL of whole blood isolated from a subject, e.g., a human subject. In some embodiments, the collection of cells comprises a sufficient number of T cells and antigen presenting cells (APCs), (e.g., dendritic cells (DCs)) such that the collection of cells may be equally distributed into a plurality of individual biological samples. In some embodiments, the collection of cells comprises a sufficient number of T cells and antigen presenting cells (APCs), (e.g., dendritic cells (DCs)) such that the collection of cells may be equally distributed into a plurality of individual biological samples. In some embodiments, the collection of cells comprises a sufficient number of APCs and T cells isolated from a subject, e.g., a human subject, such that the collection of cells may be equally distributed into a plurality of individual biological samples, each comprising APCs and T cells (e.g., DCs and T cells) in APC:T cell ratio of about 1:1, about 1:5, or about 1:10, e.g. wherein each sample comprises at least about 5×10³, 5×10⁴, or 5×10⁵ DCs and about 5×10³, 1×10⁴, 2.5×10⁴, 5×10⁴, 1×10⁶, 2.5×10⁵, 5×10⁵, 1×10⁶, 2.5×10⁶, or 5×10⁶ T cells, e.g., the collection of cells can be derived from about 5 mL, about 10 mL, about 15 mL, about 20 mL ,or about 50 mL of whole blood isolated from a subject, e.g., a human subject.

A biological sample isolated from a subject may further be diluted with saline, buffer or a physiologically acceptable diluent. Alternatively, a biological sample from a subject may be concentrated by conventional means. A biological sample isolated from a subject may also be divided into two or more aliquots to form a “plurality of biological samples,” wherein each of the plurality of biological samples comprises about the same number of biologically active cells (e.g., T cells) and about the same amount of a supporting reagent. Accordingly, unless otherwise specified, a “plurality of biological samples” as used herein refers to a plurality of distinct populations of biologically active cells, wherein each population of biologically active cells is isolated from the same subject, comprises about the same number of biologically active cells, and is maintained in similar culture conditions, e.g., with a supporting reagent, that support the viability, activation, and/or activity of the biologically active cell.

In some embodiments of the invention, a biological sample is primed ex vivo, e.g., pre-expanded, by incubation with an antigen for about a week (e.g., about 7-10 days) before in vitro re-stimulation with the antigen for about one to three days (e.g., 6-72 hours, e.g. 18-24 hours) and subsequent hashing, enrichment and/or analysis of the unique biological sample. In some embodiments of the invention, a biological sample is not primed ex vivo before in vitro re-stimulation with the antigen and subsequent hashing of the biological sample, enrichment and/or analysis of the unique biological sample. Ex vivo priming is generally not necessary for those biological samples which may have encountered the antigen while in vivo. Priming and re-stimulation protocols, including the timing for same (e.g., 7-10 days for priming and 6-72 hours, such as 18-24 hours, for re-stimulation), for biological samples comprising T cells are well-known in the art.

In some non-limiting embodiments, each of a plurality of biological samples becomes a unique biological sample by being incubated with its own unique stimulus or unique combination of stimuli (e.g., antigen or pool of antigens (e.g., T cell epitope)) and/or its own unique barcode, e.g., (hashtag oligonucleotide) for hashtagging and optional multiplexing.

“Hashtagging,” “hashing,” “tagging,” and the like as used herein comprises contacting the biologically active cell of a unique biological sample with an molecule conjugated to a unique barcode, e.g., a unique hashtag oligonucleotide (HTO), wherein the unique barcode identifies the unique characteristic of the unique biological sample, e.g., the unique antigen (e.g., a unique T cell epitope) or a lack of a unique antigen, and wherein the molecule incorporates into the cell membrane of and/or specifically binds to a cell surface marker expressed by the biologically active cell, regardless of the activation state of the biologically active cell. In some embodiments, the HTO-molecule may incorporate into any cell, e.g., any dividing cell, and/or binds a cell surface marker expressed by most or all cells (e.g., β2 microglobulin, CD298). In some embodiments, the cell marker selected is expressed by T cells regardless of activation state, (e.g., CD2, CD3, CD4, and/or CD8, etc.). In some embodiments, where two or more molecules that label a cell with an HTO in two or more different ways (e.g., one molecule may incorporate itself into the cell membrane while the other binds a marker, the two or more molecules may bind two or more different markers) are each conjugated to an HTO and are each used in a hashtagging method to tag the same unique biological sample, the two or more molecules may comprise the same barcode. In some embodiments, the two or more markers used to hashtag a unique biological sample may be the same or different markers. In some embodiments, a first unique biological marker may be tagged with a first molecule conjugated to a first unique barcode, e.g., a first HTO, a second unique biological sample is tagged with a second molecule conjugated to a second unique barcode, e.g., a second HTO, and a third biological sample is tagged with a third molecule conjugated to a third barcode, e.g., a third barcode, wherein each of the first, second, and third molecules are identical, e.g., each incorporate itself into a cell membrane or specifically bind to the same marker, however wherein each of the first, second and third molecules comprise a unique barcode that is sufficiently different that each of the first, second and third molecule may be distinguished. After washing away unbound molecules, unique biological samples that are uniquely hashed may be pooled and optionally incubated an additional reagents for further functional and phenotypic analyses of an antigen-specific activated T cell population (e.g., flow cytometric analysis and/or fluorescence cell activated sorting, single-cell sequence analysis, etc.) since the hashtagging allows for later detection, tracking and or quantitation of the each of the samples and targets that are derived from the same sample.

Some non-limiting embodiments may further enhance the sensitivity and/or robustness of a method described herein. For example, in some non-limiting embodiments, pooling 20 potentially reactive oligo-hashed assay samples per scSEQ sample typically results in adequate enrichment. In some embodiments, a combinatorial hashing approach may be taken to increase the sensitivity of the assay. For example, two or more molecules that respectively label a cell with its respective HTO in two or more different ways (e.g., one molecule may incorporate itself into the cell membrane while the other binds a marker, the two or more molecules may bind two or more different markers etc. (e.g., β2 microglobulin and CD2)), are each conjugated to the same barcode, e.g., an HTO comprising the same sequence, and are each used in a hashtagging method to tag the same unique biological sample.

Generation and use of a “hashtag oligonucleotide,” “HTO,” or the like, including the conjugation of a hashtag oligonucleotide, e.g., to a molecule (e.g., an antibody or other macromolecule, e.g., a lipid) that optionally and in some non-limiting embodiments preferably bind an activation induced marker, are well-known. See, e.g., WO2018144813; Stoeckius et al. (2018) Genome Biol. 19:224; van Buggenum J A G L et al., each of which reference is incorporated herein in its entirety by reference. Generally, an HTO comprises a unique barcode, e.g., a nucleic acid comprising a unique sequence that may be determined according to standard polymerase chain reaction protocols, e.g., single cell RNA sequencing protocols that sequence the cellular transcriptome (see, e.g., Stoeckius et al. (2017) Nat. Method 9:2579-10), which unique sequence identifies, in the embodiments described herein, a stimulus or combination of stimuli that activates a biological sample, e.g., causes the biological sample to express an activation induced marker. Conjugation chemistry, e.g., iEDDA click chemistry, may be used to conjugate, e.g., covalently attach the hashtag oligonucleotides to a molecule, e.g., a ligand that binds a cell surface marker, e.g., a constitutively expressed cell surface marker. In some embodiments, the cell surface marker is expressed by most or all cells including T cells (e.g., β2 microglobulin, CD298). In some embodiments, the cell marker is selected expressed by T cells regardless of activation state, (e.g., CD2, CD3, CD4, and/or CD8, etc.). Although oligo-tagged antibodies are described herein, other oligo-tagged tracking molecules beyond antibodies can be used, such as oligo-tagged cell membrane incorporating lipids and cell penetrating nucleic acids, particularly for further functional and/or phenotypic characterization based on single cell sequencing analysis.

A hashtag oligonucleotide (HTO) used in these compositions and methods may be conjugated any naturally occurring or synthetic biological or chemical molecule which may be used to label a cell, e.g., a lipid that incorporates into a cell membrane and/or a ligand that binds specifically to a single identified marker. The binding can be covalently or non-covalent, i.e., conjugated or by any known means taking into account the nature of the ligand and its respective target. The terms “first HTO-conjugated molecule” and “additional HTO-conjugated molecule” or “second HTO-conjugated molecule” and the like refer to HTO-conjugated molecules that label a cell in different ways, e.g., one molecule may incorporate itself into the cell membrane while the second molecule binds a marker, the two or more molecules may bind to different targets or different portions of a target. For example, multiple “first HTO-conjugated molecules” incorporate into the cell membrane or bind to the same marker at the same site. Multiple additional HTO-conjugated molecules bind to a marker different than the first HTO-conjugated molecule and different than any additional HTO-conjugated molecule. An HTO-conjugated molecule (e.g., a first HTO-conjugated molecule, and additional HTO-conjugated molecules, e.g., a second, third, fourth and fifth HTO-conjugated molecules, etc.) may independently be selected from a peptide, a protein, an antibody or antibody fragment (e.g., an antigen binding portion of an antibody), an antibody mimetic, an affibody, a ribo- or deoxyribo-nucleic acid sequence, an aptamer, a lipid, a cholesterol, a polysaccharide, a lectin, or a chimeric molecule formed of multiples of the same or different molecules. Additional non-limiting examples of HTO-conjugated molecules include those comprising a Fab, Fab′, F(ab′)2, Fv fragment, single-chain Fv (scFv), diabody (Dab), synbody, nanobodies, BiTEs, SMIPs, DARPins, DNLs, Duocalins, adnectins, fynomers, Kunitz Domains Albu-dabs, DARTs, DVD-IG, Covx-bodies, peptibodies, scFv-Igs, SVD-Igs, dAb-Igs, Knob-in-Holes, triomAbs, the like or combinations thereof. In some embodiments, a molecule conjugated to an HTO is a recombinant or naturally occurring protein. In certain embodiments, a molecule conjugated to an HTO is a monoclonal or polyclonal antibody, or fragment thereof. In one embodiment, the molecule to which the HTO is conjugated may itself also be directly labeled with one or more detectable labels, such as fluorophores that can be measured by methods independent of the methods of measuring or detecting the barcode, e.g., HTO, according to well-known methods.

In some embodiments an HTO-conjugated molecule comprises a lipid that incorporates itself into the cell membrane. In some embodiments an HTO-conjugated molecule comprises cholesterol that incorporates itself into the cell membrane. In some embodiments, an HTO-conjugated molecule comprises lipid- and cholesterol-modified oligonucleotides (LMOs and CMOs). See, e.g., McGinnis et al. (2019) Nature Methods 16:619-26, incorporated by reference in its entirety.

Assays for the further functional and phenotypic analysis of an antigen-specific activated T cell population are well-known in the art and include, but are not limited to fluorescence cell activated sorting and/or flow cytometric analysis using fluorescently labeled binding proteins (e.g., antibodies) or MHC multimers, single-cell RNA sequencing (scRNA-seq) and/or Cellular Indexing of Transcriptomes and Epitopes by sequencing (CITE-seq) analysis, etc. “Flow cytometry” encompasses methods comprising suspending cells or particles in a fluid and injected the suspension into a flow cytometer, which focuses the sample to ideally flow one cell at a time through a laser beam, where the light scattered is characteristic to the cells and their components. Cells labeled with fluorescent labels absorb the laser light and emitted in a band of wavelengths that may be used to distinguish the cells. In a preferred embodiment, after hashing and pooling, unique biological samples are enriched for activated T cells, e.g., sorted for those cells expressing activation-induced markers. In one embodiment, the cells are enriched for activated T cells, e.g., sorted, using a fluorescently labeled antibody to an activation induced marker and fluorescence activated cell sorting (FACS) prior to or simultaneous with any further functional and phenotypic analyses of the cells, e.g., prior to or simultaneous with any additional flow cytometric analyses and/or single cell sequence analysis (which may include CITE-seq analysis of any CITE-seq reagents added to the biological sample before or after the sorting). “CITE-seq” encompasses methods in which oligonucleotide-labeled molecules, e.g., oligonucleotide-labeled antibodies, are used to measure protein expression levels of a sample, e.g., during single cell sequencing approaches as described in, e.g., Stoeckius et al. (20017) Nat. Methods 14:865-868, incorporated herein in its entirety by reference. In some non-limiting embodiments, a further functional and phenotypic analysis of the cells comprises flow cytometric analysis with fluorescently labeled antibodies that detect protein expression levels of cell surface markers, e.g., additional activation markers, or intracellular proteins, e.g., intracellular cytokines. In some non-limiting embodiments, a further functional and phenotypic analysis of the cells comprises single-cell RNA sequencing of each activated cell. Non-limiting exemplary platforms for single-cell RNA sequencing include, but are not limited to plate-based approaches or microfluidic/nanowell approaches, e.g., droplet-based microfluidic approaches such as but not limited to Drop-seq (Macosko, et al. (2015) Cell 161:1202-14), InDrop (Kein et al. (2015) Cell 161:1187-1201), 10× Genomics (Zhen et al (2017) Nat. Commun. 8:1-12), and the ILLUMINA®/BIO-RAD single-cell sequencing solution. Since mRNA expression level may not correlate well with protein expression levels in a cell, in some non-limiting embodiments, single-cell RNA sequencing is performed in combination with CITE-Seq analysis, using e.g., oligonucleotide tagged antibodies, MHC multimers, and the like (see, e.g., WO2018144813, incorporated herein by reference in its entirety).

An “activation-induced marker” (AIM) is a marker that is expressed, or in which the expression is upregulated, after activation of a T cell. Well-known activation-induced markers for T cells include, but are not limited to, CD137/4-1BB, CD107, IFNγ, PD-1, CD40L, OX40, CD25, CD69, CD28, HLA-DR, CX3CR1, TIM3, LAG3, TIGIT, etc. In some embodiments, the T cell activation marker, e.g., the activation induced marker, comprises CD40L. CD40L may also be referred to as CD154. In some embodiments, the T cell activation marker, e.g., the activation induced marker, comprises CD137. CD137 is also referred to herein as 4-1BB. Accordingly, CD137/4-1BB refers to the molecule known in the art as CD137, 4-1BB, and the like, and the phrases “CD137,” “4-1BB,” and “CD137/4-1BB” may be used interchangeably. CD137/4-1BB is a transient T cell activation marker that is upregulated rapidly upon antigen-specific TCR engagement and remains expressed on cells for approximately 72 hours. In methods described herein, between 20-36 hours after exposure to an antigen appears to be the optimal time point for functional enrichment of CD137/4-1BB expression and detection. In some embodiments, the activation-induced marker comprises CD107. CD107 may also be referred to as CD107a or LAMP1. In some embodiments, the activation-induced marker comprises interferon gamma (IFNγ), which may also be referred to as gamma interferon, IFNG, IFG, etc. In some embodiments, the activation-induced marker comprises PD-1, which may also be referred to as programmed cell death 1, CD279, and HPD-1. In some embodiments, the activation-induced marker comprises TNF Receptor Superfamily member 4, which may also be referred to as OX40 and/or CD134. In some embodiments, the activation-induced marker comprises interleukin-2 receptor alpha, which may also be referred to as IL-2R, IL-2Rα, and/or CD25. In some embodiments, the activation-induced marker comprises CD69, which may also be referred to leukocyte surface antigen Leu-23 and/or MLR3. In some embodiments, the activation-induced marker comprises CD28, which may also be referred to Tp44 and/or T-cell specific surface glycoprotein. In some embodiments, the activation-induced marker comprises major histocompatibility complex class II DR, which may also be referred to as HLA-DR. In some embodiments, the activation-induced marker comprises C X C motif chemokine receptor (CX3CR1), which may also be referred to as IL-8 Receptor, IL-8Rα, and/or CDw128a. In some embodiments, the activation-induced marker comprises TIM3, which may also be referred to as Hepatitis A Virus Cellular Receptor 2, T cell Membrane Protein 3, and/or CD366. In some embodiments, the activation-induced marker comprises lymphocyte activation gene 3 (LAG3), which may also be referred to as CD223. In some embodiments, the activation-induced marker comprises T cell Immunoreceptor with Ig and ITIM Domains (TIGIT), which may also be referred to as V-Set and Immunoglobulin Domain Containing Protein 9 (VSIG9) and/or V-Set and/or Transmembrane Domain Containing 3 (VSTM3).

The terms “immunoglobulin, “antibody,” “antibodies,” “binding protein” and the like refer to monoclonal antibodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), intrabodies, minibodies, diabodies and anti-idiotypic (anti-Id) antibodies (including, e.g., anti-Id antibodies to antigen-specific TCR), and epitope-binding fragments of any of the above. The terms “antibody” and “antibodies” also refer to covalent diabodies such as those disclosed in U.S. Pat. Appl. Pub. 20070004909, incorporated herein by reference in its entirety, and Ig-DARTS such as those disclosed in U.S. Pat. Appl. Pub. 20090060910, incorporated herein by reference in its entirety.

As used herein, the term “detectable label” means a reagent, moiety or compound capable of providing a detectable signal, depending upon the assay format employed. A label may be associated with a molecule only and/or with the unique barcode (e.g., unique HTO) or a functional portion thereof. Alternatively, different labels may be used for each component of the HTO-conjugated molecule. Such labels are capable, alone or in concert with other compositions or compounds, of providing a detectable signal. In one embodiment, the labels are interactive to produce a detectable signal. In one specific embodiment, the label is detectable visually, e.g. colorimetrically. A variety of enzyme systems operate to reveal a colorimetric signal in an assay, e.g., glucose oxidase (which uses glucose as a substrate) releases peroxide as a product that in the presence of peroxidase and a hydrogen donor such as tetramethyl benzidine (TMB) produces an oxidized TMB that is seen as a blue color. Other examples include horseradish peroxidase (HRP) or alkaline phosphatase (AP), and hexokinase in conjunction with glucose-6-phosphate dehydrogenase that reacts with ATP, glucose, and NAD+ to yield, among other products, NADH that is detected as increased absorbance at 340 nm wavelength. Still other label systems that may be utilized in the described methods and molecules are detectable by other means, e.g., colored latex microparticles (Bangs Laboratories, Indiana) in which a dye is embedded may be used in place of enzymes to provide a visual signal indicative of the presence of the labeled molecule in applicable assays. Still other labels include fluorescent compounds, fluorophores, radioactive compounds or elements. In one embodiment, a fluorescent detectable fluorochrome, e.g., fluorescein isothiocyanate (FITC), phycoerythrin (PE), allophycocyanin (APC), coriphosphine-0 (CPO) or tandem dyes, PE-cyanin-5 or -7 (PC5 or PC7)), PE-Texas Red (ECD), PE-cyanin-5.5, rhodamine, PerCP, and Alexa dyes. Combinations of such labels, such as Texas Red and rhodamine, FITC+PE, FITC+PECy5 and PE+PECy7, among others may be used depending upon assay method. The selection and/or generation of suitable labels for use in labeling the molecule and/or any component of the polymer molecule is within the skill of the art, provided with this specification.

The term “specifically binds,” “binds in a specific manner,” or the like, indicates that the molecules involved in the specific binding are (1) able to stably bind, e.g., associate, e.g., form intermolecular non-covalent bonds, under physiological conditions, and are (2) unable to stably bind under physiological conditions to other molecules outside the specified binding pair.

The term “protein” encompasses all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The terms “oligonucleotide,” “nucleic acid” and “nucleotide” encompass both DNA, RNA, modified bases, or combinations of these bases unless specified otherwise. In some embodiments, a hashtag oligonucleotide comprises DNA. In some embodiments, a hashtag oligonucleotide comprises 3 to 100, 3 to 50, 3 to 30, 5 to 30, 10 to 20, 5 to 20, or 5 to 15 nucleotides. In some embodiments, a hashtag oligonucleotide comprises a sequence of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 91, 92, 93, 94, 95, 96, 97, 98, 99 or up to 100 nucleotides. In some embodiments, a hashtag oligonucleotide comprises a polyA sequence, which may comprise ten or more (e.g., 10-40, 10-30 or 10-20) consecutive adenosine nucleotides, derivatives or variants of an adenosine nucleotide.

The term “autologous” refers to biological components isolated from the same source and includes those biological components not isolated from the same source but which have physical (e.g., amino acid sequence) and functional characteristics as if the biological components were isolated from the same source. In contrast, “heterologous” refers to an agent or entity from a different source.

In accordance with the disclosure herein, there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition. Cold Spring Harbor, N.Y.: Cold Spring Harbor Laboratory Press, 1989 (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); Ausubel, F. M. et al. (eds.). Current Protocols in Molecular Biology. John Wiley & Sons, Inc., 1994, each of which publications is incorporated herein in its entirety by reference. These techniques include site directed mutagenesis, see, e.g., in Kunkel, Proc. Natl. Acad. Sci. USA 82: 488-492 (1985), U.S. Pat. No. 5,071,743, Fukuoka et al., Biochem. Biophys. Res. Commun. 263: 357-360 (1999); Kim and Maas, BioTech. 28: 196-198 (2000); Parikh and Guengerich, BioTech. 24: 4 28-431 (1998); Ray and Nickoloff, BioTech. 13: 342-346 (1992); Wang et al., BioTech. 19: 556-559 (1995); Wang and Malcolm, BioTech. 26: 680-682 (1999); Xu and Gong, BioTech. 26: 639-641 (1999), U.S. Pat. Nos. 5,789,166 and 5,932,419, Hogrefe, Strategies 14. 3: 74-75 (2001), U.S. Pat. Nos. 5,702,931, 5,780,270, and 6,242,222, Angag and Schutz, Biotech. 30: 486-488 (2001), Wang and Wilkinson, Biotech. 29: 976-978 (2000), Kang et al., Biotech. 20: 44-46 (1996), Ogel and McPherson, Protein Engineer. 5: 467-468 (1992), Kirsch and Joly, Nucl. Acids. Res. 26: 1848-1850 (1998), Rhem and Hancock, J. Bacteriol. 178: 3346-3,349 (1996), Boles and Miogsa, Curr. Genet. 28: 197-198 (1995), Barrenttino et al., Nuc. Acids. Res. 22: 541-542 (1993), Tessier and Thomas, Meths. Molec. Biol. 57: 229-237, and Pons et al., Meth. Molec. Biol. 67: 209-218; each of which publications is incorporated herein in its entirety by reference.

Methods and Compositions

The compositions and methods described herein are useful for (a) detecting the absence or presence of a functional activation of a biological sample, e.g., cell, isolated from a subject, e.g., a human subject and/or (b) identifying a stimulus and optionally the unique cognate TCR sequence.

In one embodiment, a method for identifying an antigen, e.g., T cell epitope, capable of activating a T cell, and optionally a T-cell receptor (TCR) α chain sequence and/or a TCR β chain sequence of a TCR that specifically binds the antigen, e.g., T cell epitope, described herein comprises:

(I) sorting an activated T cell, based on the expression of an activation-induced marker (AIM), from a composition comprising a unique biological sample, which unique biological sample comprises:
(a) a T cell and a surface bound Major Histocompatibility Complex (MHC), wherein the T cell is capable of recognizing a peptide presented in the context of the surface bound MHC,
(b) a unique antigen,
(c) a unique hashtag oligonucleotide (HTO) that may be used to specifically identify, preferably specifically identifies, the unique antigen, wherein the unique HTO is conjugated to a molecule that labels the T cell with the unique HTO, and optionally,
(d) medium that supports activation of the T cell,

(II) performing single cell sequencing analysis on the activated T cell sorted in (I) to identify the unique HTO conjugated to the molecule that labeled the activated T cell with the unique HTO, wherein identifying the unique HTO identifies the antigen capable of activating the activated T cell, and optionally wherein the single cell sequencing analysis also identifies one or more of the following:
(i) one or more genes expressed by the activated T cell, and/or

(ii) TCR α and/or β chain sequences of a TCR expressed by the activated T cell.

In some embodiments, the method as described herein comprises

(I) sorting one or more activated T cell(s) from a composition comprising a pool of unique biological samples, and

(II) performing single cell sequencing analysis on an activated T cell sorted in (I) to identify the antigen to which the activated T cell is reactive.

In some embodiments, each of which unique biological samples sorted in (I) comprises:

(a) a T cell and a surface bound Major Histocompatibility Complex (MHC), wherein the T cell is capable of recognizing a peptide presented in the context of the surface bound MHC, wherein each T cell of each unique biological sample is isolated from the same subject and wherein each MHC of each unique biological sample has the same haplotype (optionally wherein each MHC of each unique biological sample is derived from the same sample, bound to the same surface (e.g., a cell membrane of an antigen presenting cell), etc.),

(b) a unique antigen, e.g., T cell epitope,

(c) a unique hashtag oligonucleotide (HTO), wherein the unique hashtag oligonucleotide is conjugated to a molecule that labels the T cell with the HTO, wherein the unique HTO comprises a unique nucleotide sequence that specifically identifies the unique antigen, e.g., T cell epitope, of (b), and optionally,

(d) medium that supports activation of the T cell,

such that the single cell sequencing analysis in (II) identifies the unique HTO conjugated to the entity, wherein identifying the unique nucleotide sequence of the unique HTO identifies the antigen, e.g., T cell epitope, capable of activating the activated T cell. In some embodiments the HTO-conjugated molecule comprises a lipid that incorporates itself into the cell membrane. In some embodiments, the HTO-conjugated molecule comprises a ligand that is specifically bound to a cell surface marker expressed by the T cell. In some embodiments, the cell surface marker expressed by the T cell is ubiquitously expressed by many cells, e.g., the cell surface marker may be β2 microglobulin. In some embodiments, the cell surface marker may be selectively expressed by all T cells regardless of activation state. In some embodiments, the cell marker is selected from the group consisting of β2 microglobulin, CD298, CD2, CD3 CD4, CD8 and a combination thereof. In some embodiments, the single cell sequencing analysis also identifies one or more genes expressed by the activated T cell, and/or TCR α and/or β chain sequences of a TCR expressed by the activated T cell.

In some embodiments, the method further comprises forming a pool of unique biological samples. Forming a pool of unique biological samples may comprise creating a plurality of biological samples, e.g., by equally distributing a biological sample isolated from a subject and comprising at least a T cell and preferably an MHC (e.g., peripheral blood mononuclear cells (PBMCs), T cells and APCs, etc.) into individual samples, maintaining the biological sample in conditions that support the viability, activation and/or activity of the T cell (e.g., wherein each biological sample comprises media and cytokines that support PBMC, e.g., T cell and APC, viability and activity). As described herein, the T cells and MHC used in the methods described herein may be derived from any source. In some embodiments, the MHC are expressed on antigen presenting cells, e.g., the biological sample comprises T cells and MHC expressed on the surface of antigen presenting cells (APCs). In some embodiments, the T cells and APCs are autologous. Non-limiting and exemplary sources of APC include whole peripheral blood mononuclear cells (PBMCs), monocyte-derived dendritic cells (DCs), B cells, macrophages, normal tissue or tumor cells, APC cell lines, etc. T cells may be stimulated using co-culture of APC with T cells. In some embodiments, whole PBMC provides both the APC and T cells.

In some of these and other embodiments, the method further comprises creating a unique biological sample by delivering (i) a unique antigen, e.g., a unique T cell epitope, to a biological sample isolated from a subject and comprising at least a T cell and preferably also an MHC and/or (ii) a unique HTO conjugated to a molecule that labels the T cell with the HTO. In some embodiments, the unique biological sample is primed with the unique antigen for about 7-10 days prior to being simultaneously re-stimulated with the antigen, after which re-stimulation, the samples are hashed with a unique HTO. In some embodiments, the unique biological sample is not primed ex vivo before being simultaneously re-stimulated with the antigen and hashed with a unique HTO (e.g., the samples have been primed in vivo). In some embodiments, the samples are re-stimulated for at least 6 hours before being hashed. In some embodiments, the samples are re-stimulated for at least 16 hours before being hashed. In some embodiments, the samples are re-stimulated for at least about 18-24 hours before being hashed. In some embodiments, the samples are re-stimulated for about 48 hours before being hashed. In some embodiments, the samples are re-stimulated for about 72 hours before being hashed. In some embodiments, the samples are re-stimulated for no more than 96 hours before being hashed. In some embodiments, the method further comprises pooling unique biological samples to thereby create a composition comprising unique biological samples.

In some embodiments, sorting one or more activated T cell(s) comprises an activation induced marker (AIM) assay. In some embodiments, the AIM assay comprises fluorescence activated cell sorting of activated T cells bound to a fluorescently labeled ligand that specifically binds an activation-induced marker. Accordingly, in some embodiments, a method described herein comprises, before sorting the activated T cells from a composition comprising a pool of unique biological samples, incubating the unique biological samples with a fluorescently labeled ligand that specifically binds an activation-induced marker. The step of incubating may occur simultaneous with any hashing step and/or after pooling the unique biological samples.

In some embodiments, the fluorescently labeled ligand is a fluorescently labeled antibody and/or the activation-induced marker is selected from the group consisting of CD137/4-1BB, CD107, IFNγ, PD-1, CD40L, OX40, CD25, CD69, CD28, HLA-DR, CX3CR1, TIM3, LAG3, and/or TIGIT, and a combination thereof. In some embodiments, the activation-induced marker comprises CD137/4-1BB.

In some embodiments, the method comprises performing further functional and/or phenotypic analysis of the activated T cell. In some embodiments, the further functional and/or phenotypic analysis comprises flow cytometric analysis, CITE-seq analysis, multimer analysis, or a combination thereof. In some embodiments, the further functional and/or phenotypic analysis measures the protein and/or expression levels of one or more of CD3, CD4, CD8, CD25, CD27, CD28, CD45RA, CD62L, HLA-DR, CD137/4-1BB, CD69, CD278, CD274, CD279, CD127, CD197, IFNγ, GZMH, GNLY, CD38, CCL3, and LAG3.

Also described herein are hashed samples that are biologically active, e.g., wherein the cells are exhibiting a detectable function. In some embodiments, a composition as described herein comprises a biological sample that comprises:

(a) a T cell and a surface bound Major Histocompatibility Complex (MHC), wherein the T cell is capable of recognizing a peptide presented in the context of the surface bound MHC,

(b) an antigen, e.g., a T cell epitope,

(c) a hashtag oligonucleotide (HTO), wherein the HTO is conjugated to a molecule that labels the T cell with the HTO, and wherein the HTO comprises a nucleotide sequence that specifically identifies the antigen, e.g., T cell epitope, of (b), and optionally

(d) medium that supports activation of the T cell.

In some embodiments, the molecule that labels the T cell with an HTO is a lipid. In some embodiments, the molecule that labels the T cell with an HTO is an antibody that binds a cell marker.

Also described herein are compositions that may be used in the methods described herein. In some embodiments, a composition described herein comprises a biological sample that comprises:

(a) a T cell and a surface bound Major Histocompatibility Complex (MHC), wherein the T cell is capable of recognizing a peptide presented in the context of the surface bound MHC,

(b) an antigen,

(c) a hashtag oligonucleotide (HTO) that specifically identifies the antigen, wherein the HTO is conjugated to a molecule that labels the T cell with the HTO, and optionally

(d) medium that supports activation of the T cell.

In some embodiments, a composition comprises a pool of (e.g., at least 2) unique biological samples, e.g., a composition comprising a first and a second biological sample (and in some embodiments additional biological samples), wherein each of the first and second biological samples comprises:

(a) a T cell and a surface bound Major Histocompatibility Complex (MHC), wherein the T cell is capable of recognizing a peptide presented in the context of the surface bound MHC,

(b) an antigen, e.g., a T cell epitope,

(c) a hashtag oligonucleotide (HTO), wherein the HTO is conjugated to a molecule that labels the T cell with the HTO, and wherein the HTO comprises a nucleotide sequence that may be used to specifically identify, and preferably specifically identifies, the antigen of (b), and optionally

(d) medium that supports activation of the T cell.

In some embodiments, the second biological sample comprises

(a) a second T cell and a second surface bound MHC, wherein the second T cell is capable of recognizing a peptide presented in the context of the second surface bound MHC,

(b) a second antigen, e.g., a second T cell epitope,

(c) a second HTO, wherein the hashtag oligonucleotide is conjugated to a second molecule that labels the T cell with the HTO, and wherein second HTO comprises a second sequence that specifically identifies the second antigen, e.g., the second T cell epitope, of (b), and optionally

(d) a second medium that supports activation of the second T cell,

wherein (i) the T cell of the first sample and second T cell are isolated from the same subject, and the MHC of the first sample and second MHC are bound to the same surface, and preferably have the same haplotype (e.g., are isolated from the same source) (ii) the antigen of the first sample, e.g., the first T cell epitope, and the second antigen, e.g., the second T cell epitope, are not identical, (iii) the first molecule and the second molecule are identical, and the first nucleotide sequence of the first HTO and the second nucleotide sequence of the second HTO are not identical.

In some embodiments a composition as described herein comprises at least 2 unique biological samples. In some embodiments a composition as described herein comprises at least 3 unique biological samples. In some embodiments a composition as described herein comprises at least 4 unique biological samples. In some embodiments a composition as described herein comprises at least 5 unique biological samples. In some embodiments a composition as described herein comprises at least 6 unique biological samples. In some embodiments a composition as described herein comprises at least 7 unique biological samples. In some embodiments a composition as described herein comprises at least 8 unique biological samples. In some embodiments a composition as described herein comprises at least 9 unique biological samples. In some embodiments a composition as described herein comprises at least 10 unique biological samples. In some embodiments a composition as described herein comprises at least 11 unique biological samples. In some embodiments a composition as described herein comprises at least 12 unique biological samples. In some embodiments a composition as described herein comprises at least 13 unique biological samples. In some embodiments a composition as described herein comprises at least 14 unique biological samples. In some embodiments a composition as described herein comprises at least 15 unique biological samples. In some embodiments a composition as described herein comprises at least 17 unique biological samples. In some embodiments a composition as described herein comprises at least 18 unique biological samples. In some embodiments a composition as described herein comprises at least 19 unique biological samples. In some embodiments a composition as described herein comprises at least 20 unique biological samples. In some embodiments a composition as described herein comprises at least 30 unique biological samples. In some embodiments a composition as described herein comprises at least 50 unique biological samples. In some embodiments a composition as described herein comprises at least 80 unique biological samples. In some embodiments a composition as described herein comprises at least 100 unique biological samples

In some composition embodiments described herein, the MHC is expressed on the surface of an antigen presenting cell (APC), e.g., a dendritic cell. In some embodiments, the T cell and APC are autologous, the T cell and the APC are each isolated from a human donor, and/or the APC is a dendritic cell.

In some composition embodiments described herein, the antigen, e.g., T cell epitope, is selected from the group consisting of (i) a bacterial antigen or portion thereof, (ii) a viral antigen or portion thereof, (iii) an allergen or portion thereof, (iv) a tumor associated antigen or a portion thereof, and (v) a combination thereof In some composition embodiments described herein, the antigen, e.g., T cell epitope, comprises (i) an amino acid sequence, (ii) a nucleotide sequence, (iii) cell lysate, and (iv) a combination thereof.

In some composition embodiments described herein, the HTO is conjugated to a molecule that is an antibody and/or the molecule binds a cell surface marker selected from the group consisting of β2 microglobulin, CD298, CD2, CD3, CD4, and/or CD8.

In some embodiments, the medium comprises a cytokine that supports the viability of the T cell and/or an APC, optionally wherein the cytokine is selected from the group consisting of IL-2, IL-7, IL-15, IL-21, GM-CSF, IL-4, FLT3L, and a combination thereof. In some embodiments, the medium comprises, in lieu or in addition to the cytokine(s) that supports the viability of the T cell and/or an APC an anti-CD28 and/or anti-CD3 antibody.

In some embodiments, the biological sample comprises peripheral blood mononuclear cells (PBMCs) isolated form a subject. In some embodiments, the PBMCs are newly isolated PBMCs. In other embodiments, the PBMCs are freshly thawed PBMCs that have been cryopreserved. In some embodiments, the biological sample comprises a co-culture of dendritic cells and T cells, e.g., autologous dendritic cells and T cells.

In some embodiments, a composition described herein further comprises a fluorescently labeled antibody that specifically binds a T cell activation marker, optionally wherein the T cell activation marker is selected from the group consisting of CD137/4-1BB, CD107, IFNγ, PD-1, CD40L, OX40, CD25, CD69, CD28, HLA-DR, CX3CR1, TIM3, LAG3, and/or TIGIT, and a combination thereof. In some embodiments, a composition as described herein further comprises additional antibodies useful for flow cytometric analysis or CITE-seq analysis and/or MHC multimers (e.g., fluorescently labeled multimers and/or oligo-tagged multimers).

### Kits

The methods and compositions provided herein may be useful in high throughput assessments of immune responses. Since the methods provided herein may be utilized on patient samples regardless of MHC haplotype, also provided herein are kits for off the shelf analysis of such T cell responses.

In some embodiments, a kit comprises a plurality of unique antigens, e.g., a plurality of unique T-cell epitopes, and a plurality of unique HTO-conjugated molecules, wherein each of the plurality of unique HTO-conjugated molecules comprises a unique HTO comprising a unique HTO sequence and an identical molecule, and wherein each of the plurality of unique HTO sequences is assigned to only one of the plurality of unique antigens (e.g., one of the plurality of unique T cell epitopes) such that the unique HTO sequence may identify the unique antigen (e.g., T cell epitope) to which it is assigned. In some embodiments, each of the plurality of antigens is derived from the same source, e.g., the plurality of antigens comprises a panel of overlapping peptides from a single antigen, e.g., to aid in epitope mapping. In some embodiments, the single antigen may be a pathogenic antigen, e.g., a bacterial or viral antigen. In non-limiting embodiments, such kits comprising a plurality of antigens (e.g., T cell epitopes) derived from a pathogenic antigen which may be useful in vaccine development or in monitoring a patient's immune response to an established vaccine. In some embodiments, the single antigen may be a tumor associated antigen. In non-limiting embodiments, such kits comprising a plurality of antigens (e.g., T cell epitopes) derived from a tumor associated antigen which may be useful in immunotherapy development, e.g., in identifying the TCR variable (e.g., CDR3) sequences associated with T cell mediated cytotoxicity against tumor cells. In some embodiments, the single antigen may be an autoantigen. In non-limiting embodiments, such kits comprising a plurality of antigens (e.g., T cell epitopes) derived from an autoantigen which may be useful in monitoring a patient's autoimmune responses. In some embodiments, the single antigen may be a transplantation antigen. In non-limiting embodiments, such kits comprising a plurality of antigens (e.g., T cell epitopes) derived from a transplantation antigen which may be useful in identifying donor organs less likely to be rejected by a subject and/or establish graft versus host disease. Some kit embodiments may further comprise additional components, e.g., negative and/or positive control antigens, buffers, vials, instructions for use, multi-well culture plates, etc.

Such kits may be useful in high throughput analysis of T cell responses, e.g., (1) to potential or ongoing therapies such as vaccines, immunotherapies, etc., (2) during autoimmune disorders or transplant rejection, (3) for the development of TCR based therapeutics, and/or (4) for determining TCR:epitope binding algorithms. Accordingly, also provided herein are methods of using the high throughput screening methods, compositions and/or kits described herein for assessing immune responses and/or identifying TCR sequences (e.g., TCR variable sequences, e.g., TCR α and/or β variable sequences, e.g., TCR α and/or β CDR1, CDR2, and/or CDR3 sequences) associated with activated T cells participating in the immune responses.

### Uses

The methods and compositions provided herein may be useful for assessing immune responses. Accordingly, also described herein are methods of using the high throughput screening methods, related compositions and/or related kits for studying immunological responses in the context of T cell activation, immunological tolerance, etc.

A method described herein does not appear to affect the relative fractions of the different cell fractions, particularly the fraction of the antigen-specific T cell population, of a sample (e.g., peripheral blood mononuclear cells (PMBCs), aspirates) from the time of isolation, through any pre-stimulation or re-stimulation cultures, to the time of cell sorting. Accordingly, provided are methods of using the high throughput screening methods, compositions and/or kits described herein for evaluating the relative population sizes of antigen-specific T cells in a sample.

Also provided are methods of using the high throughput screening methods, compositions and/or kits described herein for testing vaccine candidates. In one embodiment, provided herein is a method of evaluating whether a vaccine will activate an immunological response (e.g., T cell proliferation, cytokine release, etc.) in a subject and lead to generation of effector, as well as memory T cells (e.g., central and effector memory T cells) and/or identifying the molecular phenotype of the activated immunological immune response.

The present invention also provides methods of using the high throughput screening methods, related compositions and/or kits described herein for adoptive T cell therapy. Thus, provided herein is a method of treating or ameliorating a disease or condition (e.g., a cancer) in a subject (e.g., a mammalian subject, e.g., a human subject). In some embodiments, the disease or condition is cancer. In other embodiments, the disease or condition is caused by a virus or a bacterium.

In some embodiments, the adoptive T cell therapy methods described herein comprise identifying the nucleic acid sequences encoding the TCR α and/or β variable domains, e.g., the sequences of the CDR1, CDR2, and/or CDR3 of the TCR α and/or β variable domains, (or, in other embodiments, the nucleic acid sequences encoding the TCRδ and/or γ variable domains) of antigen-specific T cells and the cognate antigen using the high throughput screening methods, compositions and/or kits described herein. In some embodiments, the nucleic acid sequences encoding the TCR α and/or β variable domains, e.g., the sequences of the CDR1, CDR2, and/or CDR3 of the TCR α and/or β variable domains, (or, in other embodiments, the nucleic acid sequences encoding the TCRδ and/or γ variable domains) identified are employed in the creation of a human therapeutic.

In one embodiment, the human therapeutic is a T cell (e.g., a human T cell, e.g., a T cell derived from a human subject) harboring a nucleic acid sequence of interest (e.g., transfected or transduced or otherwise introduced with the nucleic acid of interest) such that the T cell expresses the TCR with affinity for an antigen of interest. In one aspect, a subject in whom the therapeutic is employed is in need of therapy for a particular disease or condition, and the antigen is associated with the disease or condition. In one aspect, the T cell is a cytotoxic T cell, the antigen is a tumor associated antigen, and the disease or condition is cancer. In one aspect, the T cell is derived from the subject. Accordingly, upon identification of the nucleic acids and the cognate antigen, an adoptive T cell therapy method described herein may further comprise cloning the nucleic acid sequence of the T cell receptor or a portion thereof (e.g., nucleic acid sequence of a TCR variable domain) identified by the method described herein, into an expression vector (e.g., a retroviral vector), introducing the vector into T cells derived from the subject such that the T cells express the antigen-specific T cell receptor, and infusing the T cells into the subject.

In other embodiments of an adoptive T cell therapy described herein, the nucleic acid sequence(s) encoding the TCR α and/or β variable domains, e.g., the sequences of the CDR1, CDR2, and/or CDR3 of the TCR α and/or β variable domains, (or, in other embodiments, the nucleic acid sequences encoding the TCRδ and/or γ variable domains) of antigen-specific T cells are employed in the creation of a human T cell receptor therapeutic. In one embodiment, the therapeutic receptor is a soluble T cell receptor. Much effort has been expanded to generate soluble T cell receptors or TCR variable regions for use therapeutic agents. Generation of soluble T cell receptors depends on obtaining rearranged TCR variable regions. One approach is to design single chain TCRs comprising TCRα and TCRβ, and, similarly to scFv immunoglobulin format, fuse them together via a linker (see, e.g., International Application No. WO 2011/044186). The resulting scTv, if analogous to scFv, would provide a thermally stable and soluble form of TCRα/β binding protein. Alternative approaches included designing a soluble TCR having TCRβ constant domains (see, e.g., Chung et al., (1994) Functional three-domain single-chain T-cell receptors, Proc. Natl. Acad. Sci. USA. 91:12654-58); as well as engineering a non-native disulfide bond into the interface between TCR constant domains (reviewed in Boulter and Jakobsen (2005) Stable, soluble, high-affinity, engineered T cell receptors: novel antibody-like proteins for specific targeting of peptide antigens, Clinical and Experimental Immunology 142:454-60; see also, U.S. Pat. No. 7,569,664). Other formats of soluble T cell receptors have been described. The method described herein may be used to determine a sequence of a T cell receptor that binds with high affinity to an antigen of interest, and subsequently design a soluble T cell receptor based on the sequence.

A soluble T cell receptor comprising the sequences identified according to the high throughput methods, compositions, and/or kits described herein may be used to block the function of a protein of interest, e.g., a viral, bacterial, or tumor associated protein. Alternatively, a soluble T cell receptor may be fused to a moiety that can kill an infected or cancer cell, e.g., a cytotoxic molecules (e.g., a chemotherapeutic), toxin, radionuclide, prodrug, antibody, etc. A soluble T cell receptor may also be fused to an immunomodulatory molecule, e.g., a cytokine, chemokine, etc. A soluble T cell receptor may also be fused to an immune inhibitory molecule, e.g., a molecule that inhibits a T cell from killing other cells harboring an antigen recognized by the T cell. Such soluble T cell receptors fused to immune inhibitory molecules can be used, e.g., in blocking autoimmunity. Various exemplary immune inhibitory molecules that may be fused to a soluble T cell receptor are reviewed in Ravetch and Lanier (2000) Immune Inhibitory Receptors, Science 290:84-89, incorporated herein by reference.

Non-limiting and exemplary embodiments are provided below.
Embodiment 1. A method for identifying T-cell receptor (TCR) α and/or β chain sequences of a TCR that recognizes an epitope of interest comprising sorting from a pool of T cells a population of T cells labeled with an oligonucleotide conjugated antibody, wherein the oligonucleotide tag comprises a sequence associated with a unique epitope, antigen, or antigen pool.
Embodiment 2. The method of embodiment 1, comprising after sorting, the step of determining the sequence of the oligonucleotide tag to thereby determine the epitope, antigen, or antigen pool that activated the T cell labeled with the oligonucleotide conjugated antibody.
Embodiment 3. The method of embodiment 1 or embodiment 2, comprising one or more of the following steps prior to the sorting step
   creating a plurality of cultures from a peripheral blood mononuclear cells (PBMC) sample, e.g., in a multi-well culture plate, wherein each culture comprises media and cytokines that support antigen presenting cell (APC) and T cell function and growth,
   delivering to each one of the plurality of cultures a unique antigen or antigen pool of interest thereby creating a unique culture, e.g., adding a single antigen (or antigen pool) of interest into one of the plurality of cultures, e.g., one well of the culture plate, wherein each culture (well) comprises a unique antigen or antigen pool,
   adding to a unique culture a unique oligonucleotide tag that is associated with the unique culture, and
   optionally adding other surface staining antibodies and multimers, which may also comprise an oligomeric tag, e.g., CITE-seq and oligo-dextramer reagents, and
   pooling the cultures.
Embodiment 4. The method of any one of embodiments 1-3, comprising
   creating a plurality of cultures from a peripheral blood mononuclear cells (PBMC) sample, e.g., in a multi-well culture plate, wherein each culture comprises media and cytokines that support antigen presenting cell (APC) and T cell function and growth.
   delivering to each one of the plurality of cultures a unique antigen or antigen pool of interest thereby creating a unique culture, e.g., adding a single antigen (or antigen pool) of interest into one of the plurality of cultures, e.g., one well of the culture plate, wherein each culture (well) comprises a unique antigen or antigen pool,
   adding to a unique culture an antibody that binds a T cell activation marker in sufficient amounts to label all cells present in each well, and a molecule tagged with a unique oligonucleotide tag that is associated with the unique culture, and
   optionally adding other surface staining antibodies and multimers, which may also comprise an oligomeric tag, e.g., CITE-seq and oligo-dextramer reagents,
   pooling the cultures.
   sorting those T cells that are labeled with the antibody that binds a T cell activation marker and tagged with a unique oligonucleotide tag, and
   determining nucleic acid sequences from the T cells sorted in step (5), including the nucleic acid sequences of the unique oligonucleotide tags.
Embodiment 5. The method of any one of embodiments 1-4, wherein the T cell activation marker comprises CD137/4-1BB.
Embodiment 6. A method for identifying an antigen capable of activating a T cell, and optionally a T-cell receptor (TCR) α chain sequence and/or a TCR β chain sequence of a TCR that specifically binds the antigen, the method comprising:
   (I) sorting an activated T cell, based on the expression of an activation-induced marker (AIM) from a composition comprising a unique biological sample, which unique biological sample comprises:
      (a) a T cell and a surface bound Major Histocompatibility Complex (MHC), wherein the T cell is capable of recognizing a peptide presented in the context of the surface bound MHC,
      (b) a unique antigen,
      (c) a unique hashtag oligonucleotide (HTO) that may be and/or is used to specifically identify, e.g., a unique HTO that specifically identifies, the unique antigen, wherein the unique HTO is conjugated to a molecule that labels the T cell with the unique HTO, and optionally,
      (d) medium that supports activation of the T cell,
   (II) performing single cell sequencing analysis on the activated T cell sorted in (I) to identify the unique HTO conjugated to the molecule that labeled the activated T cell with the unique HTO, wherein identifying the unique HTO identifies the antigen capable of activating the activated T cell, and optionally wherein the single cell sequencing analysis also identifies one or more of the following:
   (i) one or more genes expressed by the activated T cell, and/or
   (ii) TCR α and/or β chain sequences of a TCR expressed by the activated T cell.
Embodiment 7. The method of embodiment 6, comprising, before sorting, one or both of the following step(s):
   creating a plurality of biological samples by equally distributing a collection of cells comprising T cells and antigen presenting cells (APCs) isolated from a subject into individual samples, wherein each biological sample optionally comprises media and cytokines that support T cell and/or APC viability, activation and/or activity, and
   creating a plurality unique biological samples by delivering to each of a plurality of biological samples a unique antigen and/or a unique HTO that may be and/or is used to specifically identify, e.g., a unique HTO that specifically identifies, the unique antigen, wherein the unique HTO is conjugated to a molecule that labels a T cell with the unique HTO, and optionally combining the plurality of unique biological samples such the composition sorted in (I) comprises a plurality of unique biological samples
      wherein each of the plurality of biological samples comprises a collection of cells comprising T cells and APCs isolated from a subject and optionally medium that supports viability, activity, and/or activation of the T cells and APCs, and
      wherein after delivery of the unique antigen and/or the unique HTO conjugated to a molecule that labels a T cell with the unique HTO, each of the plurality of biological samples becomes a unique biological sample that comprises
         (a) a collection of cells comprising T cells and APCs isolated from a subject,
         (b) a unique antigen,
         (c) a unique HTO that specifically identifies the unique antigen and is conjugated to a molecule that labels the T cell with the HTO, and optionally
         (d) medium that supports viability, activity, and/or activation of the T cells and APCs.
Embodiment 8. The method of embodiment 7, wherein the APCs comprise monocyte-derived dendritic cells, dendritic cells, monocytes, macrophages, B cells, or a combination thereof.
Embodiment 9. The method of any one of embodiments 6-8, wherein sorting comprises fluorescence activated cell sorting of activated T cells based on the expression of the activation-induced marker (AIM).
Embodiment 10. The method of embodiment 9, wherein the AIM is selected from the group consisting of CD137/4-1BB, CD107, IFNγ, PD-1, CD40L, OX40, CD25, CD69, CD28, HLA-DR, CX3CR1, TIM3, LAG3, TIGIT, and any combination thereof.
Embodiment 11. The method of embodiment 9 or embodiment 10, wherein fluorescence activated cell sorting is based on detection with fluorescently labeled antibody to the AIM.
Embodiment 12. The method of any one of embodiments 6-11, comprising performing further functional and/or phenotypic analysis on the activated T cell analyzed in II, optionally wherein the further functional and/or phenotypic analysis is selected from the group consisting of flow cytometric analysis, CITE-seq analysis, multimer analysis, and a combination thereof.
Embodiment 13. The method of embodiment 12, wherein the further functional and/or phenotypic analysis measures the protein and/or RNA expression levels of one or more of CD3, CD4, CD8, CD25, CD27, CD28, CD45RA, CD62L, HLA-DR, CD137/4-1BB, CD69, CD278, CD274, CD279, CD127, CD197, IFNγ, GZMH, GNLY, CD38, CCL3, and LAG3.
Embodiment 14. The method of any one of embodiments 6-13, wherein peripheral blood mononuclear cells provide the T cell and surface bound MHC.
Embodiment 15. The method of any one of embodiments 6-14, wherein the molecule that labels the T cell with the unique HTO comprises an antibody that binds a cell surface molecule.
Embodiment 16. The method of any one of embodiments 6-15, wherein the AIM is or comprises CD137/4-1BB.
Embodiment 17. The method of any one of embodiments 6-16, wherein the method comprises identifying a TCR α chain sequence and/or a TCR β chain sequence of a TCR that specifically binds the antigen and the TCR α chain sequence and/or a TCR β chain sequence are a TCR α chain variable region sequence and/or a TCR β chain variable region sequence, respectively.
Embodiment 18. The method of any one embodiments 6-17, wherein the method comprises identifying a TCR α chain sequence and/or a TCR β chain sequence of a TCR that specifically binds the antigen and the method further comprises utilizing the TCR α chain sequence and/or a TCR β chain sequence in making a therapeutic.
Embodiment 19. A composition comprising a biological sample that comprises:
   (a) a T cell and a surface bound Major Histocompatibility Complex (MHC), wherein the T cell is capable of recognizing a peptide presented in the context of the surface bound MHC,
   (b) an antigen,
   (c) a hashtag oligonucleotide (HTO) that specifically identifies the antigen, wherein the HTO is conjugated to a molecule that labels the T cell with the HTO,
   and optionally
   (d) medium that supports activation of the T cell.
Embodiment 20. The composition of embodiment 19, wherein
   (a) the MHC is expressed on the surface of an antigen presenting cell (APC), optionally wherein:
      the T cell and APC are autologous,
      the T cell and the APC are each isolated from a human donor, and/or
      the APC is selected from the group consisting of a monocyte-derived dendritic cell, a dendritic cell, a monocyte, a macrophage, a B cell and a combination thereof,
   (b) the antigen
      (I) is selected from the group consisting of
         (i) a bacterial antigen or portion thereof,
         (ii) a viral antigen or portion thereof,
         (iii) an allergen or portion thereof,
         (iv) a tumor associated antigen or a portion thereof, and
         (v) a combination thereof, and/or
      (II) comprises
         (i) an amino acid sequence,
         (ii) a nucleotide sequence,
         (iii) lysate, and
         (iv) a combination thereof,
   (c) the HTO conjugated molecule comprises
      (I) an antibody that binds a cell surface molecule, or
      (II) a lipid, and/or
   (d) the medium comprises a cytokine that supports the viability of the T cell and/or an APC.
Embodiment 21. The composition of embodiment 20, wherein
   the antibody binds a cell surface marker selected from the group consisting of β2 microglobulin, CD298, CD2, CD3, CD4, CD8, and any combination thereof, or
   the lipid that incorporates into a cell membrane.
Embodiment 22. The composition of embodiment 20 or embodiment 21, wherein the cytokine that supports the viability of the T cell and/or the APC is selected from the group consisting of IL-2, IL-7, IL-15, GM-CSF, IL-4, and any combination thereof.
Embodiment 23. The composition of any one of embodiments 20-22, further comprising a second biological sample, wherein the second biological sample comprises:
   (a) a second T cell and a second surface bound MHC, wherein the second T cell is capable of recognizing a peptide presented in the context of the second surface bound MHC,
   (b) a second antigen,
   (c) a second HTO that specifically identifies the second antigen, wherein the HTO is conjugated to a second molecule that labels the second T cell with the second HTO,
   and optionally
   (d) a medium that supports activation of the second T cell,
   wherein
   (i) the T cell and second T cell are isolated from the same subject,
   (ii) the antigen and the second antigen are not identical,
   (iii) the molecule that labels the T cell with the HTO and the second molecule that labels the second T cell with the second HTO are identical, and the HTO and the second HTO are not identical.
Embodiment 24. The composition of any one of embodiments 19-23, wherein the composition further comprises an agent that allows sorting an activated T cell based on expression of an activation-induced marker (AIM).
Embodiment 25. The composition of embodiment 24, wherein the agent that allows sorting activated T cell based on expression of an AIM is a fluorescently labeled antibody that specifically binds the AIM.
Embodiment 26. The composition of embodiment 24 or embodiment 25, wherein the AIM is selected from the group consisting of CD137/4-1BB, CD107, IFNγ, PD-1, CD40L, OX40, CD25, CD69, CD28, HLA-DR, CX3CR1, TIM3, LAG3, and/or TIGIT.
Embodiment 27. The composition of any one of embodiments 19-26, wherein the composition comprises an antibody and/or MHC multimers useful for flow cytometric analysis or CITE-seq analysis of the composition.
Embodiment 28. A kit comprising
   a plurality of unique antigens, and
   a plurality of unique hashtag oligonucleotides (HTOs), each of which specifically identifies only one of the plurality of unique antigens.
Embodiment 29. The kit of embodiments 28, wherein the kit further comprises an agent that allows sorting of activated T cells based on their expression of an activation-induced marker (AIM), optionally wherein the agent that allows sorting activated T cell based on expression of an AIM is a fluorescently labeled antibody that specifically binds the AIM.
Embodiment 30. The kit of embodiments 28 or 29, wherein each of the plurality of unique HTOs is conjugated to an identical molecule such that the kit comprises a plurality of unique HTO-conjugated molecules.
Embodiment 31. The kit of any one of embodiments 28-30, wherein each of the plurality of unique antigens comprises unique and overlapping peptide sequences from a single protein.
Embodiment 32. The kit of embodiment 31, wherein the single protein is selected from the group consisting of a pathogenic antigen, a tumor associated antigen, or a transplantation antigen.
Embodiment 33. Use of the method of any one of embodiments 1-18, the composition of any one of embodiments 19-27, or the kit of any one of embodiments 28-32 for analyzing a T cell mediated immune response of a patient to a vaccine.
Embodiment 34. Use of the method of any one of embodiments 1-18, the composition of any one of embodiments 19-27, or the kit of any one of embodiments 28-32 for analyzing a T cell mediated immune response of a patient to an immunotherapy.
Embodiment 35. Use of the method of any one of embodiments 1-18, the composition of any one of embodiments 19-27, or the kit of any one of embodiments 28-32 for analyzing a T cell mediated immune response in a patient during immunotherapy of the patient.
Embodiment 36. Use of the method of any one of embodiments 1-18, the composition of any one of embodiments 19-27, or the kit of any one of embodiments 28-32 for analyzing T cell responses of a patient to an autoantigen.
Embodiment 37. Use of the method of any one of embodiments 1-18, the composition of any one of embodiments 19-27, or the kit of any one of embodiments 28-32 for analyzing T cell responses of a patient to a transplant antigen.
Embodiment 38. Use of the method of any one of c embodiments 1-18, the composition of any one of embodiments 19-27, or the kit of any one of embodiments 28-32 to identify one or more TCR variable region sequences of an activated T cell.
Embodiment 39. The use of embodiment 38, wherein the one or more TCR variable region sequences comprises a CDR3 sequence of a TCRα chain and/or a CDR3 sequence of a TCRβ chain.
Embodiment 40. Use of the one or more TCR variable region sequences identified in embodiments 38 or 39 in making a human therapeutic.
Embodiment 41. The use of embodiment 40, wherein the human therapeutic comprises a T cell comprising the one or more TCR variable region sequences identified using the method of any one of embodiments 1-18, the composition of any one of embodiments 19-27, or the kit of any one of embodiments 28-32.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims.

### EXAMPLES

A non-limiting embodiment of a method described herein is illustrated in FIG. 1. The examples herein provide data that a hashing methodology may be used in combination with a functional assay, such as activation-induced marker (AIM) cell enrichment and single cell transcriptome sequencing to screen cognate T cell and antigen reactivities, e.g., T cell epitope reactivities, and that such method finds particular usefulness in primary human cells.

General methods are provided here before more specific and exemplary applications of the methods are described.

General Materials and Methods

Human Peripheral Blood Mononuclear Cells (PBMCs): Cryopreserved PBMCs were purchased (Precision for Medicine Frederick, Md.) or isolated from fresh blood from human subjects isolated by density gradient centrifugation using Ficoll-Paque Plus (GE Healthcare Life Sciences, 45-001,749) reagent as per manufacturer's instructions and cryopreserved in freezing media (90% human serum (Millipore Sigma), 10% tissue culture-grade DMSO (Millipore Sigma, 2438) for later analysis.

Peptides: Peptides were custom synthesized at Genscript (Piscataway, N.J.). Lyophilized peptides were reconstituted in DMSO at 10-50 mg/mL for stock solutions and then further diluted to 10 μg/mL in the appropriate assay medium for use. The CEF Control Peptide Pool (Anaspec, AS-61036-003) was used at 10 ug/mL and Cell Stimulation Cocktail (ThermoFisher, 00-4970-93) as per manufacturer's instructions.

Primary Cell Cultures: Cryopreserved PBMCs were thawed and incubated in CellGenix GMP DC serum-free media (CellGenix, 20801-0500) with 5% Human Serum AB (Millipore Sigma, H3667) and 1% penicillin-streptomycin (ThermoFisher Scientific, 15140163). Cultures were supplements with dendritic cell and T cell supportive cytokines: T cell media (CellGenix dendritic cell media, cat #20801-0500+5% human serum AB (Sigma, cat #H3667))+1% penicillin/streptomycin/L-glutamine (ThermoFisher, cat #10378-016), the T cell supporting cytokines IL-7 and IL-15 at 5 ng/ml (CellGenix, cat #1410-050 and 1413-050, respectively), and IL-2 at 10 U/ml (Peprotech, cat #200-0).

Generation of oligo-tagged hashing antibodies: Monoclonal antibodies that are highly specific for cell surface targets on all human T cells (CD2, RPA-2.10; Biolegend Cat #300202)) were custom conjugated to an assortment of unique 15-base oligonucleotide sequences with polyA tails using published methods. See, e.g., Stoeckius 2017, bioRxiv, supra.

Direct ex vivo IFNγ/GranzymeB ELISPOT: Dual Human IFNγ/GranzymeB FluoroSpot assays kits were purchased from ImmunoSpot (Cleveland, Ohio) and used per manufacturer's protocol. Briefly, PBMCs were thawed and incubated in 200 uL in the FluoroSpot plates at 200,000 cells per well with peptide stimulation for 48 hours. ELISPOT reactivity was read out on an ImmunoSpot Analyzer using manufacturer's automated software.

Antibodies and phenotypic characterization of T cells by flow cytometry: Fluorescently labeled antibodies were purchased from commercial vendors. To perform flow cytometry phenotypic characterization of surface proteins, cells were harvested, washed, and resuspended in flow cytometry BD BSA staining buffer (BD Biosciences, #554657) containing antibodies of interest. Cells were incubated for 30 minutes at 4° C. and then washed twice before flow cytometry acquisition on an A3 Symphony cytometer (BD Biosciences). Flow cytometry data were analyzed using the FlowJo analysis software (FlowJo, Ashland, Oreg.). Gates were set based in fluorescent minus one (FMO) controls.

Antigen-specific T cell reactivity assays: Peripheral blood mononuclear cells (PBMC) were isolated by Ficoll-Paque Plus gradient isolation. PBMC were seeded to culture plates, e.g., aliquoted, in T cell media (CellGenix GMP DC media, cat #20801-0500+5% human serum AB (Sigma, cat #H3667))+1% penicillin/streptomycin/L-glutamine (ThermoFisher, cat #10378-016), dendritic cell supporting factors GM-CSF at 1000 U/mL and IL-4 at 500 U/mL (CellGenix, #1412-050 and CellGenix, #1403-050, respectively), T cell supporting cytokines IL-7 and IL-15 at 5 ng/ml (CellGenix, #1410-050 and 1413-050, respectively), and IL-2 at 10 U/ml (Peprotech, cat #200-0). Individual antigens, e.g., peptides of interest, were added to assay wells at 10 ug/ml (Genscript) to form unique biological samples.

Overnight cultures were harvested 24 hours post-peptide stimulation and prepared for sorting and single cell sequencing. For 10-day pre-expansion cultures, cells were fed with fresh media and cytokines every two days for one week after the initial peptide addition. Then, individual peptides of interest were added to T cell expansion cultures for overnight re-stimulation to upregulate expression of an activation-induced marker e.g., CD137/4-1BB, and enable antigen-specific AIM based functional T cell sorting. Following peptide re-stimulation, cells were prepared for either flow cytometry characterization or were further processed to enable hashing, pooling, and single cell sequencing.

Cell hashing following functional T cell assay performance: Following functional stimulation, cells from individual assay wells were collected into a 96 well assay block, washed, and resuspended in flow cytometry BD BSA staining buffer (BD Biosciences, #554657) containing hashing reagents of interest. Cells were either stained with one or two hashtag oligonucleotide (HTO) antibodies, each at 1 μg/10⁶ cells. Cells were incubated for 30 minutes at 4° C., washed twice, then pooled. If oligonucleotide-tagged dextramers were included in the analysis, then samples were stained with dextramers before proceeding to CITE-seq and flow cytometry antibody staining as per the oligo-tagged dextramer staining protocol below.

CITE-seq antibody staining and fluorescent antibody staining: Following the hashing staining procedure, pooled and hashed samples were resuspended in BD BSA staining buffer containing both CITE-seq antibodies as well as fluorescently tagged flow cytometry antibodies at their respective optimal concentrations. Cells were incubated for 30 minutes at 4° C., washed twice, and then sorted for single cell sequencing.

Oligo-tagged dextramer staining and FACS sorting: Cryopreserved health donor PBMC were thawed briefly in a 37° C. water bath. CD8+ T cells were enriched using magnetic beads (Miltenyi Biotec). Cells were washed by centrifugation and then treated with PBS (Gibco, 14190-250) containing benzonase (Millipore, 70664) and 50 nM Dasatinib (Axon Medchem, 1392) for 45 minutes at 37° C. Cells were transferred to a 96-well assay block (Corning, 3960), centrifuged, and supernatant was aspirated. The appropriate custom Immudex dCODE-PE dextramer pool (Copenhagen, Denmark) was added at 1 ul/100 ul reaction for 30 minutes in dark at room temperature. Next, the fluorochrome-labeled surface markers were added, and the cells were incubated for additional 30 minutes in 4° C. After washes, the cells were immediately sorted. Flow cytometry antibody staining and washes were performed in staining buffer (BD, 554657). Surface markers for FACS included the following markers and fluors: Live/Dead—DAPI added on-site at the sorter (Sigma, 10236276001), CD3 BUV737 (BD Biosciences, 612750), CD4 BV510 (BD Biosciences, 563919), CD8 BUV805 (BD Biosciences, 612889), CCR7 AF647 (BioLegend 353218), and CD45RO BV605 (BioLegend 304238).

CD137/4-1BB+ T cell FACS sorting: Twenty-four hours following re-stimulation, cells were collected and stained with fluorescently-labeled antibodies for FACS using an Astrios cell sorter (Beckman Coulter) using the following surface antibodies: CD3 (BD Biosciences, cat #612750) and CD137/4-1BB (Biolegend, cat #309828). Gates for forward scatter plot, side scatter plot, and fluorescent channels were set to select live cells while excluding debris and doublets. A 100 μm nozzle was used to sort single CD3+CD137/4-1BB+ cells for further processing.

Chromium single cell partitioning and library preparation: Sorted cells were then loaded onto a Chromium Single Cell 5′ Chip (10× Genomics, 1000287) and processed through the Chromium Controller to generate GEMs (Gel Beads in Emulsion). We prepared RNA-Seq libraries with the Chromium Single Cell 5′ Library & Gel Bead Kit (10× Genomics, 1000265) following the manufacturer's protocol.

Bioinformatic Methods

The transcriptome, TCR (VDJ), hashing, CITE-seq, and dextramer libraries were sequenced and the raw sequencing data was processed using the 10× CellRanger analysis pipeline. The CellRanger analysis generated feature-barcode UMI count matrices and TCR(VDJ) amino acid sequences. The features include gene expression, hashing antibody, CITE-seq antibody, and dextramer capture. Using the feature-barcode matrices as the input, the R package Seurat v3.1.4 (Butler et al 2018) was used for downstream analysis. Standard log normalization of gene UMI counts was performed, followed by identification of 1000 most variable genes, and scaling and centering of the data. Next Principal Component Analysis (PCA) was performed and 50 PCs were computed and stored. Clustering was then performed using Seurat's graph-based clustering approach. A k-nearest neighbor (KNN) graph was computed based on the Euclidean distance in a 20-dimensional PCA space followed by clustering at various resolutions. At each resolution, top marker genes were identified and used to create a heatmap of gene expression across different clusters. Upon visual inspection, the optimal clustering resolution was determined. All the cells belonging to the dead cell cluster, with mitochondrial genes as the top gene markers, were removed from the downstream analysis. Cells for which number of genes detected was less than or equal to 500, and fraction of mitochondrial gene expression was greater than or equal to 0.25 were removed. Since one of the main goals of the assay is to identify T-cell reactivity against various antigens which are driven by TCR-antigen interactions, any cell with a single TCR chain, or a non-productive chain, or more than one alpha or beta chain was also removed. Any outlier cell with large number of genes detected and/or a large number of UMIs detected was also removed. For the remaining cells, data from other features (CITE-seq, hashing, dextramer) was then processed. The data from count matrices corresponding to those features was normalized using centered log ratio transformation, and then scaled. Hashing data was used to demultiplex the cells using the MultiSeqDemux algorithm (McGinnis et al. (2019) Nature Methods 16:619-26; default parameters). Any cell that was not assigned a hashtag according to the hashing scheme was removed after multiplexing. For each cell, paired TCR amino acid sequence which defines the unique functional clonotype of the cell was obtained. After demultiplexing, the clonotype size of each T-cell clone among all the cells associated with a hashtagged assay well was calculated. Any clonotype with size>20 was considered to have potential reactivity to the specific antigen in the hashtagged well.

### Example 1

### Identifying CD137/4-1BB as an Activation Induced Marker (AIM) that is Able to Functionally Identify Antigen-Specific T Cell Populations Comparable to Multimer Staining

Materials and Methods

Generally, in the methods described in this example, T cells from a healthy HLA-A*0201+human donor were pre-expanded in the presence of cognate synthetic peptides as per the methods described herein and then stained with fluorescently-tagged antibodies and dextramer multimers for flow cytometry analysis to identify antigen-specific T cell populations.

Results

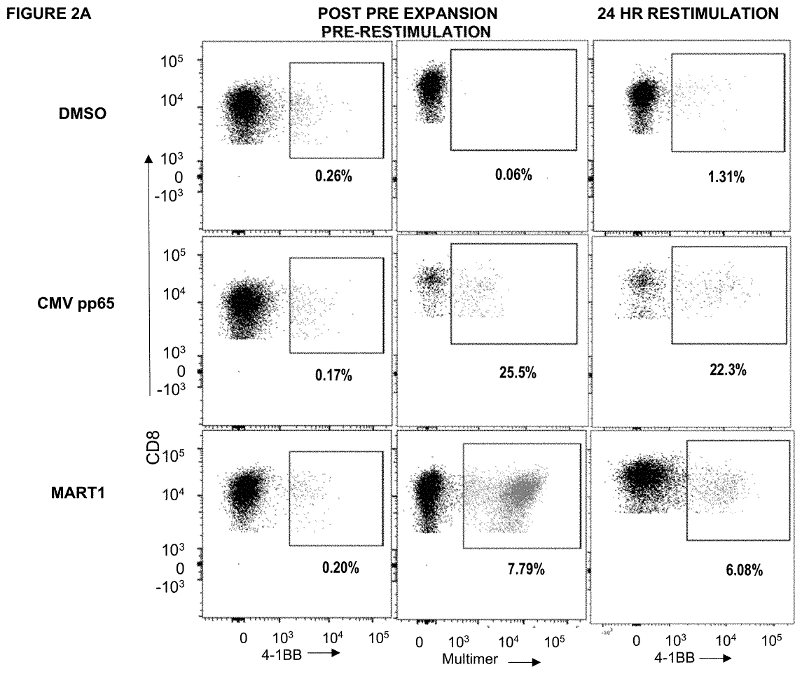

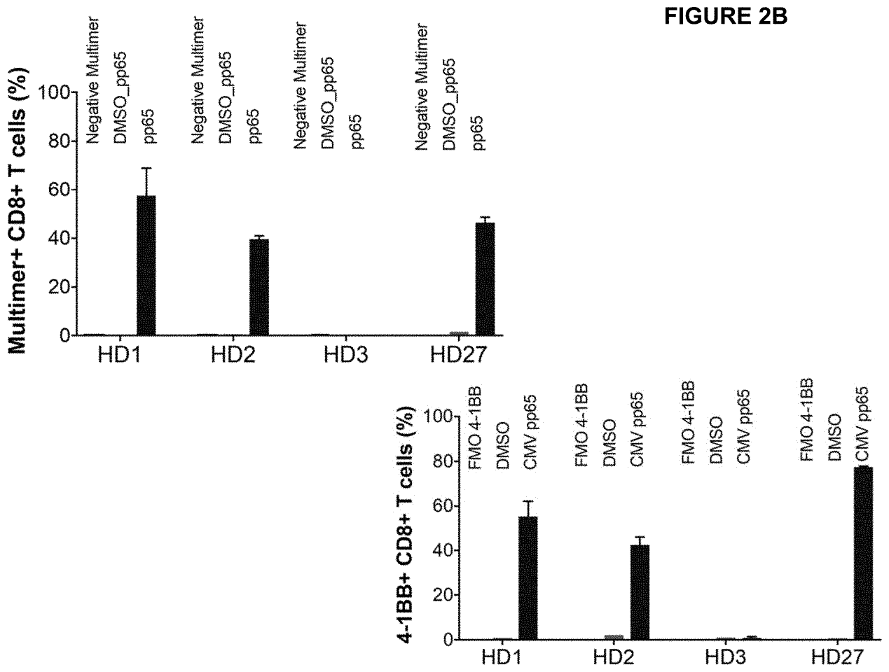

In FIG. 2A dendritic cells (DCs) were derived from whole peripheral blood mononuclear cells (PBMC) from a healthy human donor. In brief, CD14+ monocytes were isolated from PBMC by magnetic selection using anti-CD14-magnetic beads (Miltenyi). The CD14+ cells were cultured for 5 days in CellGenix CellGro DC media supplemented with IL-4 and GM-CSF. On day five, DCs were pulsed with CMV pp65 (NLVPMVATV; SEQ ID NO:16), or MART1 (ELAGIGILTV; SEQ ID NO:15) synthetic short peptides specific for HLA-A*0201 for 2 hours. Then, IFNα was added to the cells to activate them. On day 7, autologous T cells were added to the culture and the media was exchanged with CellGenix CellGro media supplemented with 5% human serum plus supportive cytokines (IL7, IL-15, IL-2). These autologous DC and T cells were cultures for 10 days to expand the relevant pre-existing antigen-specific T cell populations. After 10-day pre-expansion in culture, the T cells were re-stimulated for 24 hours with relevant peptides or a DMSO negative control. Cell surface targets of interest were assessed using fluorescently-tagged monoclonal antibodies and dextramer multimers by flow cytometry characterization (A3 Symphony analyzer, BD).

The flow cytometry dot plots of FIG. 2A demonstrate that the fraction of CD137/4-1BB+CD8+ T cells from cultures is low prior to stimulation (x-axis, left panel) but multimers robustly stain CD8+T cell populations of interest: 25.5% CMV pp65 CD8+T cells, 7.79% MART1+ T cells. (x-axis, middle panel). In other words, the cell culture conditions used in this particular example expanded pre-existing memory T cells, but did not induce de novo T cell expansion. However, following re-stimulation with cognate peptide for 24 hours, CD137/4-1BB expression is upregulated on CD8+ T cells and the total size of the CD137/4-1BB+ population (x-axis) is similar to the multimer+ population (right panel).

In FIG. 2B, using the same cell culture and staining methods as described in this example, cells isolated from 4 HLA-A*0201+healthy donors (HD1, HD2, HD3, and HD27) were cultured for 10 days in the presence of DMSO or CMV pp65 synthetic peptide. The fraction of CMVpp65 multimer+ CD8+ T cells following 10-day expansion relative to a negative control multimer was assessed by flow cytometry (FIG. 2B, top panel). The expression of CD137/4-1BB on CD8+ T cells following 24-hour re-stimulation with CMV pp65 synthetic peptide relative to DMSO control was also assessed by flow cytometry (FIG. 2B, bottom panel). Three of the four healthy donors who were seropositive for CMV had measurable CMV pp65+ CD8+ T cells (HD1, HD2, HD27) while the CMV seronegative donor (HD3) did not have detectable CMV pp65+ T cells. (FIG. 2B). Generally, there is agreement in the population size of multimer+ CD8+ and CD137/4-1BB+ CD8+ T cells. (FIG. 2B).

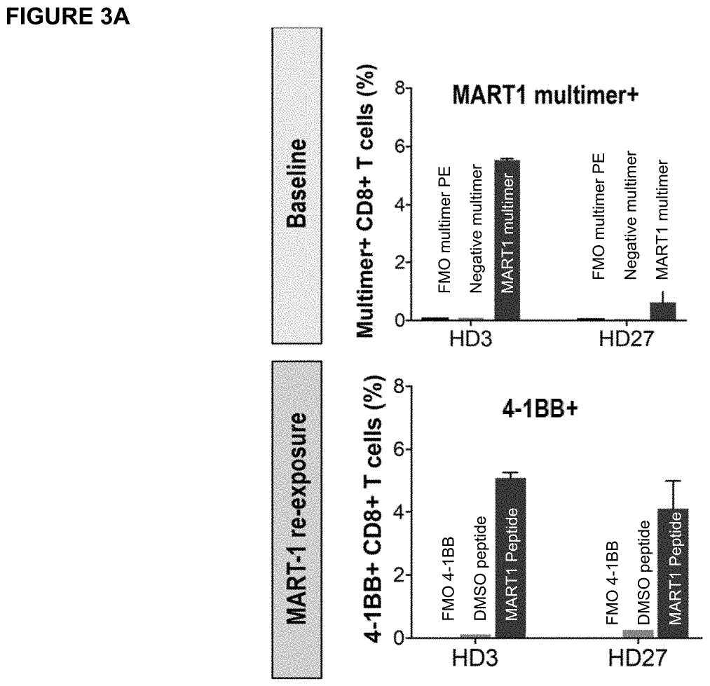

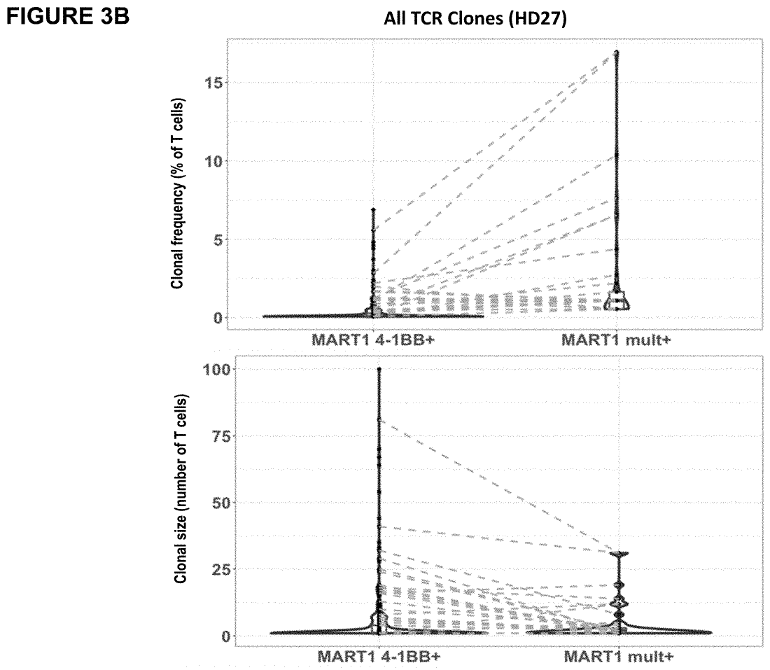

In FIG. 3A-3B, whole peripheral blood mononuclear cells (PBMCs) from healthy HLA-A*0201+human donors (HD3 and HD27) were cultured in media comprising supportive cytokines (GM-CSF, IL-4, IL7, IL-15, IL-2), and DMSO or MART1 (ELAGIGILTV; SEQ ID NO:15) synthetic short peptides or for 10 days to respectively provide a baseline population (DMSO) or expand relevant pre-existing MART1-specific T cells. After 10-day pre-expansion in culture, the T cells were re-stimulated for 24 hours with a DMSO negative control or MART1 peptide. The MART1 multimer+ CD8+ T cells and CD137/4-1BB CD8+ T cells from healthy donor 27 (HD27) were sorted by fluorescence activated cell sorting (FACS) and encapsulated in 10× Genomics single cell partitioners for 5′ RNA and TCR single cell sequencing library preparation followed by high-throughput next generation sequencing. Only cells that produced complete, paired alpha and beta TCR information were evaluated. The overlap across multimer+ and CD137/4-1BB+ samples was assessed.

Prior to re-stimulation, both donors had detectable MART1+ CD8+ T cells (FIG. 3A, top panel). Following 24-hour re-stimulation with cognate peptide, both donors (HD3 and HD27) upregulated CD137/4-1BB on their cell surface (FIG. 3A, bottom panel). However, one donor (HD27) had markedly higher CD137/4-1BB+ T cells than multimer+ CD8+ T cells (FIG. 3A). To test this discrepancy, the functional T cell clones identified by multimer and CD137/4-1BB staining were further evaluated by assessing the overlap across multimer+ and CD137/4-1BB+ samples. There was significant overlap in the TCR sequences shared between the multimer+ and CD137/4-1BB+ CD8+ T cell populations. (FIG. 3B). Overall, the CD137/4-1BB+ fraction contained more clonal populations than the MART1 multimer+ population. The most clonally expanded, MART1 multimer+ TCRs were detected in the CD137/4-1BB+ CD8+ T cell population, and many lower abundance TCRs were also detectable across both enriched populations. However, the CD137/4-1BB+ population captured TCRs that were not detected in the multimer+ population. Additionally, many low abundance TCR sequences from the MART1 multimer+ population were present as larger clone sizes in the CD137/4-1BB+ population.

The data shown in FIGS. 2A-B and 3A-B demonstrate the activation-induced marker CD137/4-1BB is upregulated on human T cells following antigen-specific activation and that there is significant overlap among the single cell paired alpha/beta chain T cell receptor (TCR) sequences between multimer+ and CD137/4-1BB+ CD8+ T cells cultured according to the methods described herein. As such, CD137/4-1BB may be used in functional assays, e.g., as a functional enrichment activation-induced marker (AIM) for antigen-specific T cells. Additionally, use of CD137/4-1BB as a functional marker is as efficient and provides similar functional assay results as traditional multimer staining.

### Example 2

### Characterizing Cognate T Cell and Epitope Reactivities in Primary Human Cells Using Hashtag Oligonucleotides and CD137/4-1BB Enrichment of Activated T Cells

To further validate hashing, AIM sorting and/or single cell sequencing analysis as a viable method to evaluate and characterize cognate antigen and TCR reactivities, unique biological samples comprising PBMCs and unique viral peptides were hashed with hashtag oligonucleotides conjugated anti-CD2 antibodies and pooled. Functional activation was identified by CD137/4-1BB staining and use of CD137/4-1BB in a functional assay was compared to conventional functional assays of ELISPOT and dextramer staining.

Materials and Methods

ELISPOT: PBMCs from a healthy HLA-A*0201+human donor with known seropositivity to CMV, EBV, and Influenza were plated in a Dual Human IFNγ/GranzymeB FluoroSpot assays plate (ImmunoSpot, Cleveland, Ohio) at a concentration of 2×10⁵ cell per well with DMSO or individual HLA-A*0201+-restricted viral peptide stimulation (EBV YVL-9, CMV pp65, EBV LMP2A, EBV BMLF1, Influenza A) for 48 hours. Following incubation, ELISPOT reactivity was developed and read on an ImmunoSpot Analyzer using manufacturer's instructions and automated software.

PBMC culture for hashing and AIM enrichment: Whole peripheral blood mononuclear cells (PBMC) from a healthy HLA-A*0201+human donor were cultured in media, supportive cytokines (GM-CSF, IL-4, IL7, IL-15, IL-2), and individual HLA-A*0201+-restricted viral peptides (EBV YVL-9, CMV pp65, EBV LMP2A, EBV BMLF1, Influenza A) for 10 days to expand the relevant pre-existing antigen-specific T cell populations. After 10-day pre-expansion in culture, the T cells were re-stimulated for 24 hours with relevant peptides or a DMSO negative control. Cell surface targets of interest were assessed using fluorescently-tagged monoclonal antibodies by flow cytometry characterization (A3 Symphony analyzer, BD). The relative fraction of CD137/4-1BB+ CD8+ T cells across viral peptide stimuli were assess by flow cytometry (CD8+ CD137/4-1BB+ T cell fractions provided as a percentage of total CD8+ T cells above the gate).

Hashing. AIM enrichment, and single cell sequencing: Monoclonal human anti-CD2 antibodies labeled with unique hashtag oligonucleotides were added to each assay well of the PBMC biological samples described in this experiment to uniquely barcode the T cells from a given well with the stimulus that same well received. Then, all assay well samples were pooled and stained with fluorescent-tagged surface antibodies for FACS sorting and CITE-seq antibodies for scSEQ phenotyping. The CD137/4-1BB+ CD8+ T cell population was sorted and demultiplexed, e.g., analyzed by single cell sequencing (10× Genomics 5′ RNA and TCR). Expression is normalized using LogNormalize method which normalizes gene expression of each cell by the total expression. Mathematically, normalized expression is equal to log 1p(UMI.Count*scaling.factor/(Total UMI count)), where scaling.factor=10,000 and log 1p is log

Oligo-tagged dextramer activation and staining CD8+ T cells were enriched using Miltenyi CD8+ T cell negative enrichment (Miltenyi). The cells were then incubated for 45 minutes with benzonase (Millipore) and dasatinib (Axon) before being stained with oligo-tagged dextramer pools for 30 minutes at room temperature. Cells were then stained with fluorescently labeled for CD3 (BD Biosciences, cat #612750), CD4 (BD Biosciences, cat #563919, CD8 (BD Biosciences, cat #612889), CCR7 (Biolegend, cat #353218), and CD45RO (Biolegend, cat #304238) and CITE-seq antibodies for an additional 30 minutes on ice. Utilizing an Astrios cell sorter (Beckman Coulter), fluorescence activated cell sorting (FACS) gating on forward scatter plot, side scatter plot, and fluorescent channels was set to select live cells while excluding debris and doublets. A 100 μm nozzle was used to sort single CD3+CD8+dextramer+ cells for further processing.

RNA Sequencing Clustering RNA transcript expression was evaluated on CD137/4-1BB+ T cells sorted from AIM enrichment. Clustering was performed using Seurat's graph-based clustering approach using a k-nearest neighbor (KNN) graph and was computed based on the Euclidean distance in a 20-dimensional PCA space followed by clustering at various resolutions.

Results

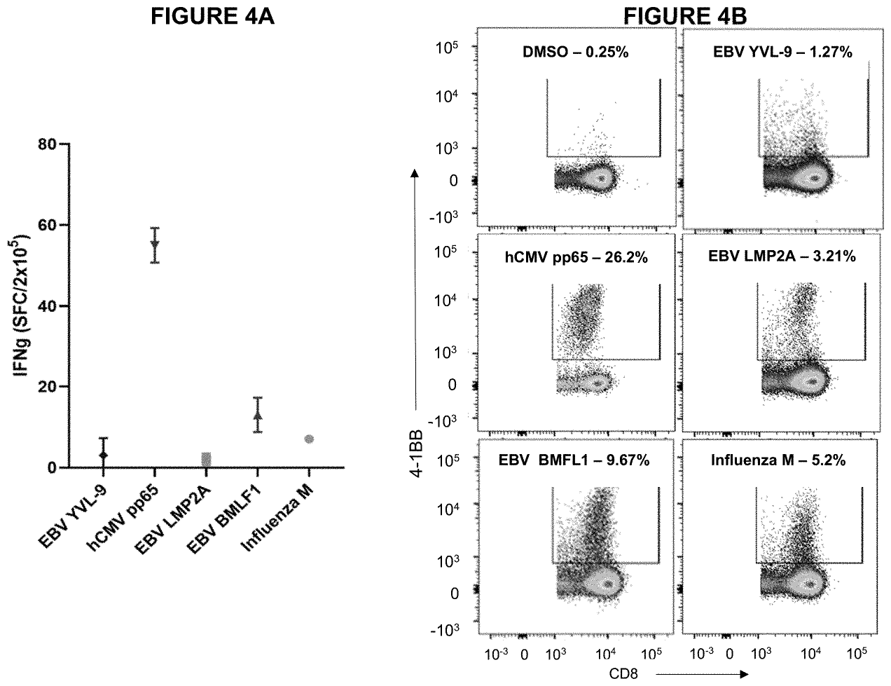

As shown in FIGS. 4A and 4B, the percentage of antigen-specific cells within a biological sample is identified by hashing and AIM enrichment (FIG. 4B) and it correlates with the percentage identified with the conventional ELISPOT functional assay (FIG. 4A). Accordingly, it appears the pre-expansion and re-stimulation protocols described herein maintain relative fractions of antigen-specific T cell populations.

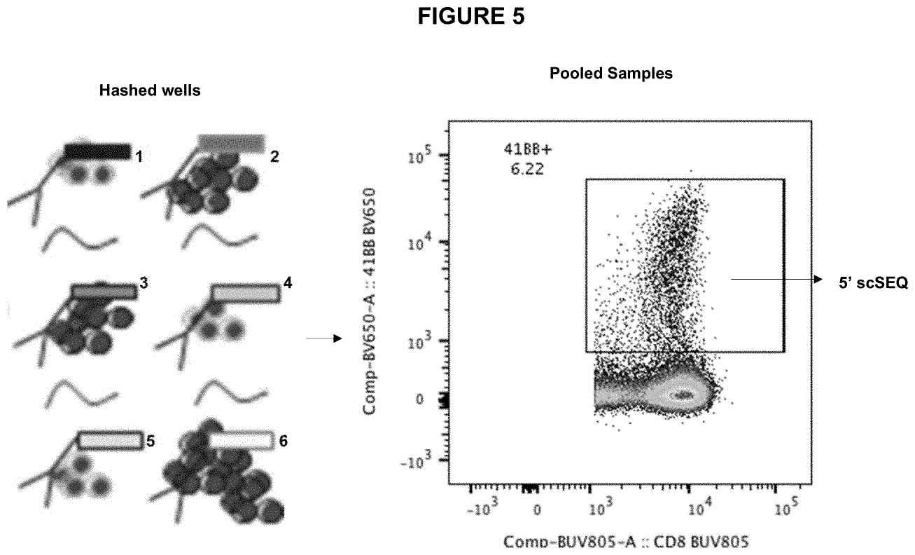

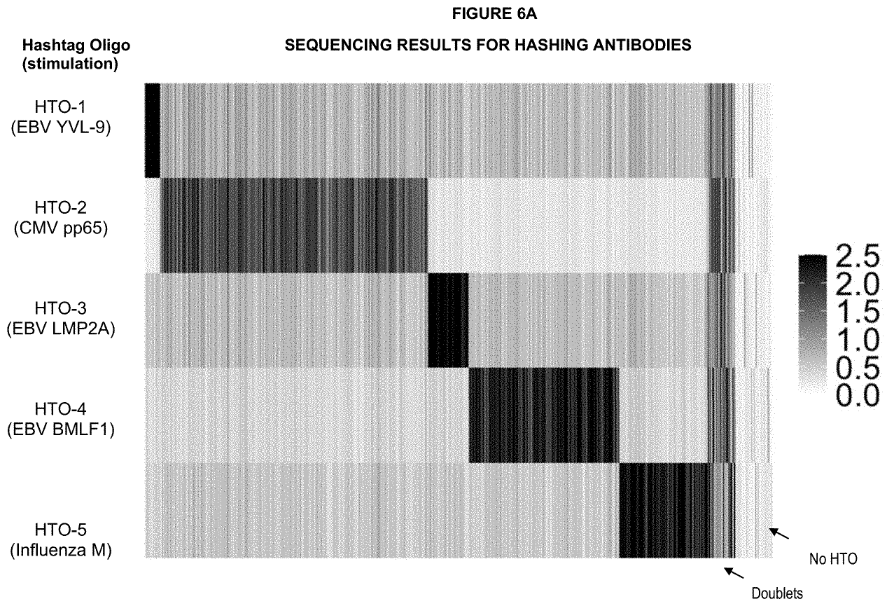

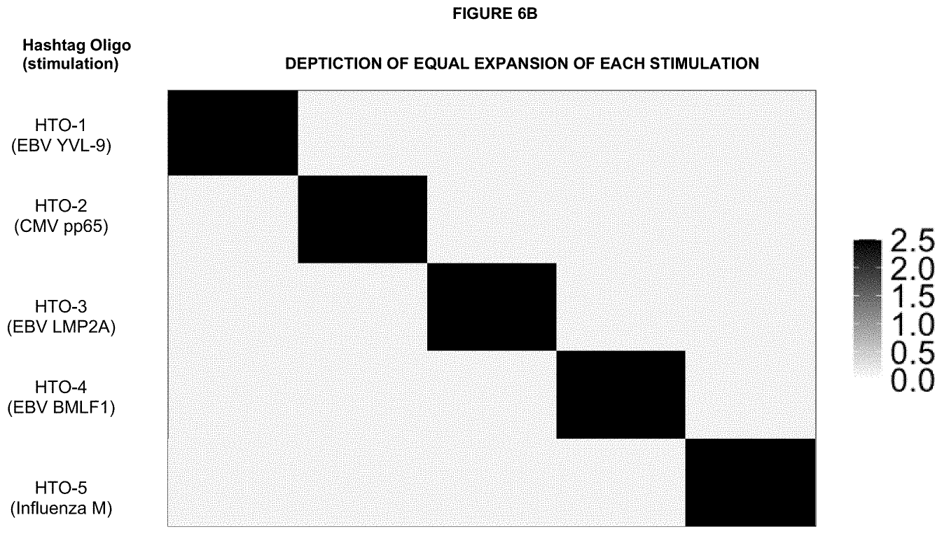

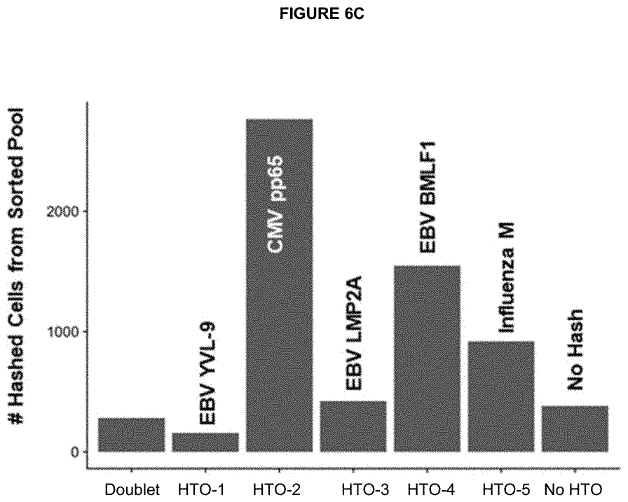

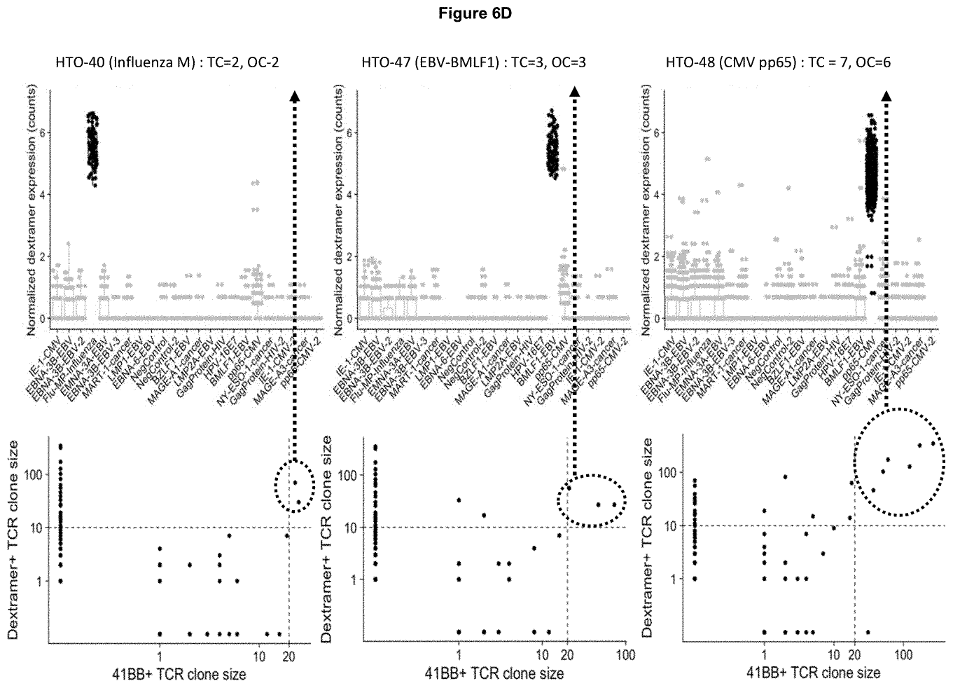

Validation of the methodologies provided herein is shown in FIG. 6. Single cell sequence analysis of the antigen-specific cells enriched and analyzed by the methods disclosed herein, a non-limiting embodiment of which is illustrated in FIG. 5, provides further validation. As shown in FIGS. 6A-C, single cell sequence analysis assigned most cells to individual HTO (80%), while ˜8% were classified as “Doublets,” and “No HTO” were identified for ˜12% of cells. The relative numbers of cells corresponding to each HTO, and thus each reactivity (EBV YVL-9, CMV pp65, EBV LMP2A, EBV BMLF1, Influenza M), mirrored the relative numbers of cells identified in orthogonal functional assays including the ELISPOT assay shown in FIG. 4A and flow cytometric analysis shown in FIG. 4B. Shown in FIG. 6B is the relative number of cells that would have been identified by each hashtag had unrealistic equal expansion of each stimulation occurred.

The reactivity of CD137/4-1BB⁺ T cells hashed to CMV pp65, EBV BMLF1, or Influenza M clones was confirmed by a parallel oligotagged pooled dextramer based experiment. FIG. 6D shows that the HTO-40, HTO-47 and HTO-48 hashed CD137/41BB expanded TCRs show reactivity for the antigens identified by the hashtags, i.e., Influenza M (HTO-40), EBV-BMLF1 (HTO-47), and pp65-CMV (HTO-48). This is observed by the presence of T-cell clones with clone size>=20 in the hashtagged wells corresponding to those antigens. The number of unique clones is denoted by total clones (TC). The reactivity of these expanded clones is confirmed by a parallel oligotagged pooled dextramer based experiment. The number of CD137/4-1BB expanded clones that are identical to the clones in the dextramer experiment with clone size>=10 is denoted by OC. These overlapping clones (OC) show high expression of dextramer corresponding to the hashed antigen and low expression of dextramer corresponding to irrelevant antigen.

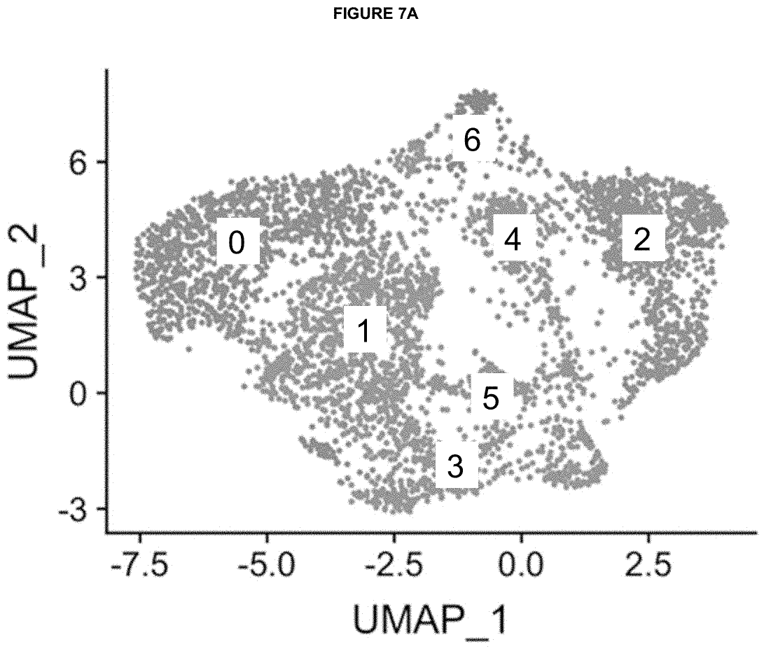

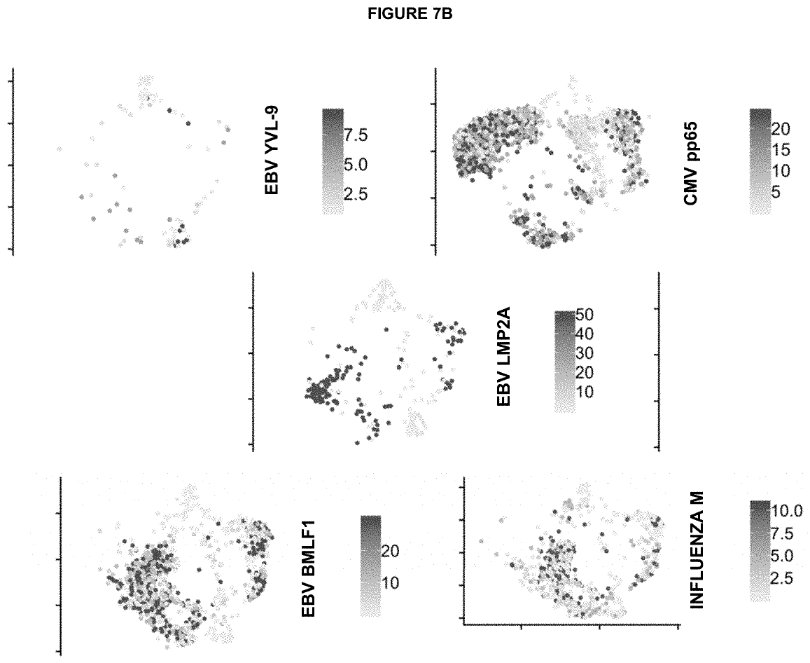

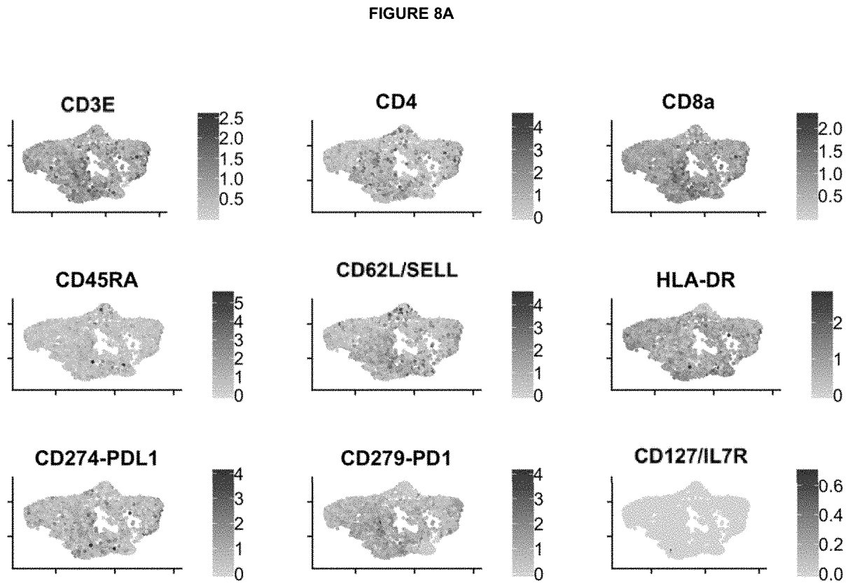

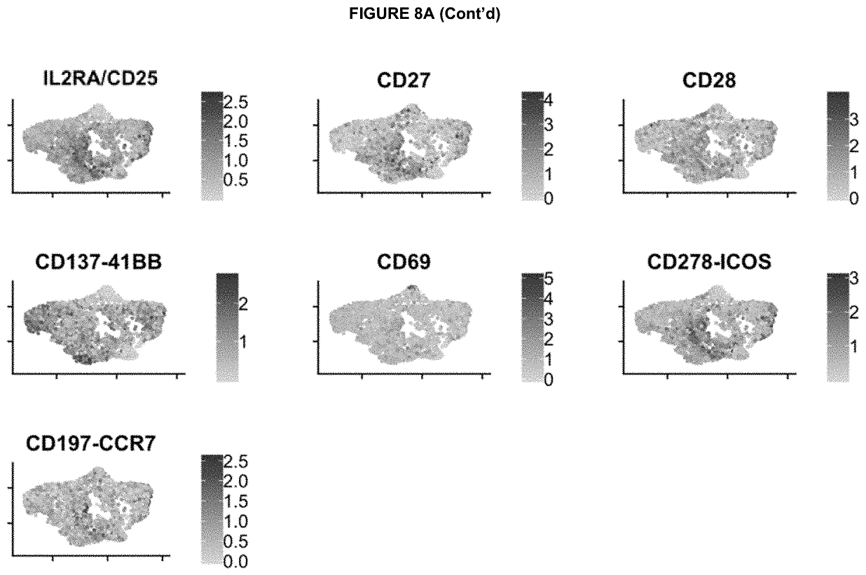

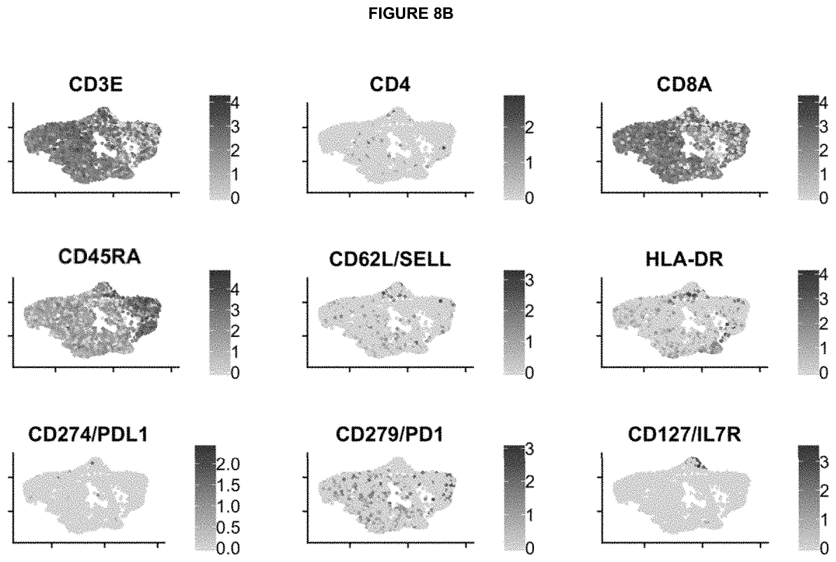

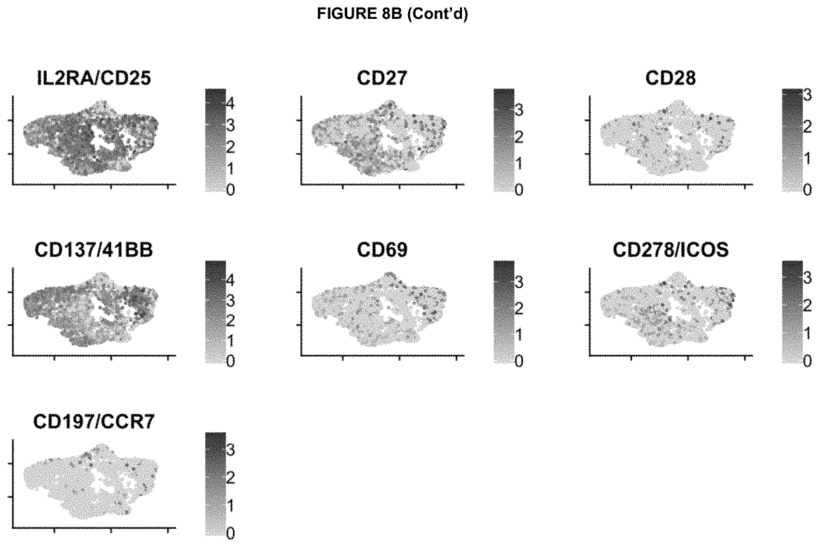

Further validation is provided in demultiplexing of scSEQ data. Seven unique clusters were resolved from the RNA transcriptome analysis based on the gene expression patterns and levels by individual cells. (FIG. 7A). Notably, comparison of the population size for each antigen-specific T cell found by demultiplexing scSEQ data (FIG. 7B) agrees with the population size of antigen-specific T cells found from functional flow cytometry assays (FIG. 4B). The data show that unique antigen-specific T cells were identifiable by HTO demultiplexing of scSEQ data and the relative population size for each antigen-specific T cell population was maintained following demultiplexing. Moreover, CITE-seq reagents are compatible with the hashing, AIM sorting, and single cell sequence analysis methodologies described herein. Use of such CITE-seq reagents may add a critical layer of information to improve cell subset identification and phenotyping. As a non-limiting example, CITE-seq data gives a measurement of protein abundance on the surface of each cell whereas RNA-seq data gives a measurement of transcript abundance in each cell. Protein abundance and RNA-seq expression may not be correlated, therefore both measurements provide complementary information. This is highlighted by comparison of the CD4 CITE-seq data shown in FIG. 8A and the CD4 RNA-seq data shown in FIG. 8B.

### Example 3

### Functional and Phenotypic Analysis of Antigen-Specific T Cells

Described herein is the use of hashing, AIM sorting, single cell sequencing, and CITE-seq antibody staining for the functional and phenotypic analysis of antigen-specific T cells performed directly on PBMCs, e.g., without a 7-10 day pre-expansion.

Materials and Methods

5′ Human TCR α/β with Cell Surface Antibody Staining: Cell Partitioning, Library Preparation, and Sequencing

Single cells suspended in PBS with 0.04% BSA were loaded on a Chromium Single Cell Instrument (10× Genomics). RNAseq, V(D)J, and antibody-derived-tag libraries were prepared using Chromium Single Cell 5′ Library, Gel Beads & Multiplex Kit (10× Genomics), with antibody-derived-tag primer addition. After amplification, cDNA was split into small (<300 bp) and large (>300 bp) fragment fractions. RNAseq and V(D)J libraries were prepared from the >300 bp fraction; cell surface antibody-derived libraries were prepared from the <300 bp fraction. To enrich the V(D)J library aliquot for TCR α/β, the cDNA was split into two 20 ng aliquots and amplified in two rounds using primers.

Specifically, for first round amplification, the primers used were MP147 (ACACTCTTTCCCTACACGACGC; SEQ ID NO:17) for short R1, MP120 (GCAGACAGACTTGTCACTGGA; SEQ ID NO:18) for human TRAC, and MP121 (CTCTGCTTCTGATGGCTCAAACA; SEQ ID NO:19) for human TRBC. For second round amplification, 20 ng aliquots from the first round were amplified using MP147, MP128 (GTGACTGGAGTTCAGACGTGTGCTCTTCCGATCTGCAGGGTCAGGGTTCTGGATA; SEQ ID NO:20) a nested R2 plus human TRAC, and MP129 (GTGACTGGAGTTCAGACGTGTGCTCTTCCGATCTGCAGGGTCAGGGTTCTGGATA; SEQ ID NO:21) a nested R2 plus human TRBC. V(D)J libraries were prepared from 25 ng each hTRAC and hTRBC amplified cDNA. Paired-end sequencing was performed on Illumina NextSeq500 for RNAseq and antibody-derived tag libraries (Read 1 26-bp for UMI and cell barcode, 8-bp i7 sample index, and Read 2 55-bp transcript read) and V(D)J libraries (Read 1 150-bp, 8-bp i7 sample index, and Read 2 150-bp read.

Results

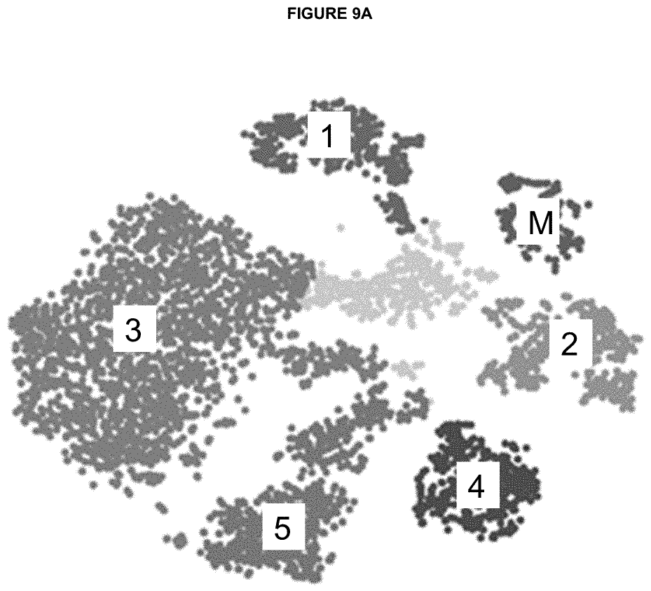

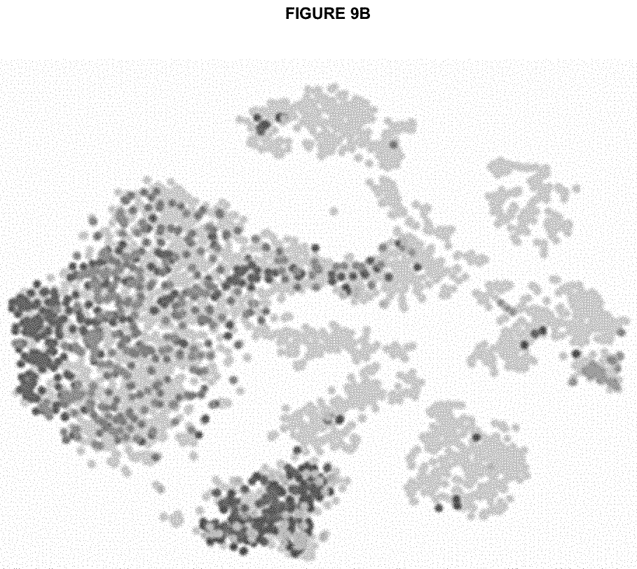

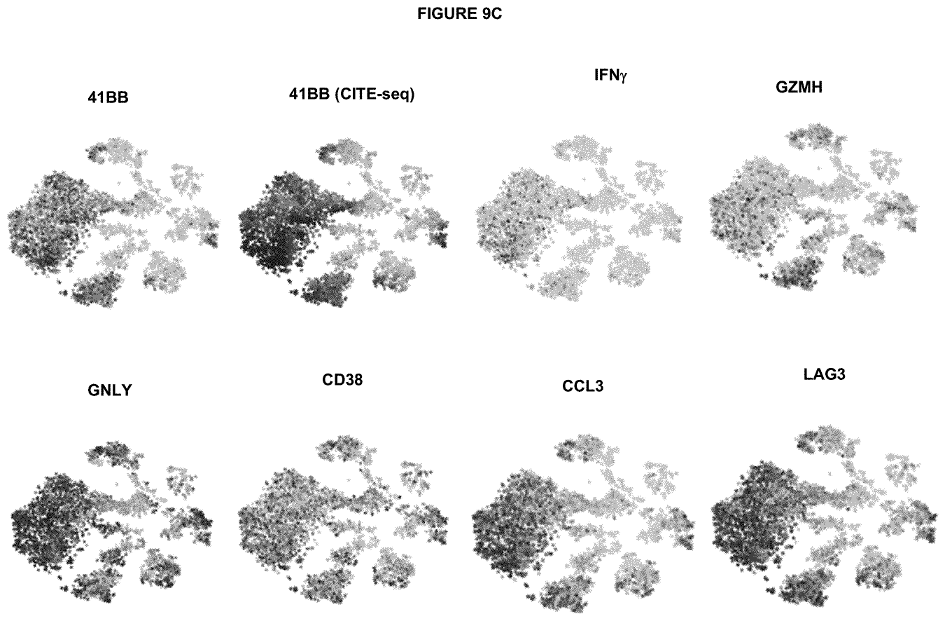

PBMCs isolated from a donor were incubated with one of five unique HPV peptides. Antigen specific T cells were clustered based on HTO sequence using AIM sorting based on CD137/4-1BB and single cell sequence analysis (FIG. 9A). Cells that represent TCR clones that are not shared across HTO samples (above positive signal threshold) were identified and the TCR sequences of these clones obtained (See, e.g., FIG. 9B). FIG. 9B provides an exemplary illustration by which each unique cell clone is represented by a different color in gray scale, where each cell of a clone is depicted by the same color in grayscale. The number of hashtag-restricted clones, i.e., the number of clones that are associated with only one HTO, for each hashtag and the number of cells in each clone is provided in Table 1 below.

TABLE 1

| Hashtag | Number of unique clones | Number of cells |
|---|---|---|
| HTO-1 | 168 | 233 |
| HTO-2 | 176 | 209 |
| HTO-3 | 416 | 1128 |
| HTO-4 | 188 | 225 |
| HTO-5 | 179 | 342 |

As shown in Table 1, cells identified by HTO-3 show the greatest number of TCR clones that express TCR specific for a cognate antigen, followed by cells clustered to HTO-5. Clones are identified by amino acid sequence, and exemplary CDR3 sequences of TCR α and β pairs of some HTO-3 restricted TCRs are provided in Table 2 below.

Table 2

| | α CDR3 amino acid sequence (SEQ ID NO:) |
|---|---|
| Hashtag | β CDR3 amino acid sequence (SEQ ID NO:) |
| HTO-3 | CAGGGSGNTGKLIF (SEQ ID NO: 1) |
| | CASSVRSSYEQYF (SEQ ID NO: 2) |
| HTO-3 | CAGGGSQGNLIF (SEQ ID NO: 3) |
| | CASSIRSSYEQYF (SEQ ID NO: 4) |
| HTO-3 | CVVWGSQGNLIF (SEQ ID NO: 5) |
| | CASSIRSSYEQYF (SEQ ID NO: 6) |
| HTO-3 | CAGGGSSNTGKLIF (SEQ ID NO: 7) |
| | CASSIRSSYEQYF (SEQ ID NO: 8) |
| HTO-3 | CAMRGSPGGTSYGKLTF (SEQ ID NO: 9) |
| | CASSEYSNQPQHF (SEQ ID NO: 10) |
| HTO-3 | CAYMTNAGGTSYGKLIF (SEQ ID NO: 11) |
| | CASSQGSYGTF (SEQ ID NO: 12) |
| HTO-3 | CAGALGGGSQGNLIF (SEQ ID NO: 13) |
| | CASSVRSSYEQYF (SEQ ID NO: 14) |

Single cell sequencing analysis shows that those T cell clones that are hash restricted, i.e., not shared across HTO samples, more highly express markers associated with a functional T cell response. (FIG. 9C; see also FIG. 9B).

Shown herein is a unique method that rapidly identifies the unique amino acid sequence of a T cell receptor that specifically binds an antigen as well as provides phenotypic characteristics of the cell that expresses the antigen-specific T cell receptor sequences. With this high throughput method, novel and potentially personalized therapeutics may be quickly identified and generated.

### Equivalents

Those skilled in the art will recognize, or be able to ascertain, using no more than routine experimentation, many equivalents of the specific embodiments of the invention described herein. Such equivalents are intended to be encompassed by the following claims.

Entire contents of all non-patent documents, patent applications and patents cited throughout this application are incorporated by reference herein in their entirety.

1. A method for identifying an antigen capable of activating a T cell, and optionally a T-cell receptor (TCR) α chain sequence and/or a TCR β chain sequence of a TCR that specifically binds the antigen, the method comprising:
(I) sorting an activated T cell, based on the expression of an activation-induced marker (AIM) from a composition comprising a unique biological sample, which unique biological sample comprises:
(a) a T cell and a surface bound Major Histocompatibility Complex (MHC), wherein the T cell is capable of recognizing a peptide presented in the context of the surface bound MHC,
(b) a unique antigen,
(c) a unique hashtag oligonucleotide (HTO) that is used to specifically identify the unique antigen, wherein the unique HTO is conjugated to a molecule that labels the T cell with the unique HTO, and optionally,
(d) medium that supports activation of the T cell, and
(II) performing single cell sequencing analysis on the activated T cell sorted in (I) to identify the unique HTO conjugated to the molecule that labeled the activated T cell with the unique HTO, wherein identifying the unique HTO identifies the unique antigen of (b), and optionally wherein the single cell sequencing analysis also identifies one or more of the following:
(i) one or more genes expressed by the activated T cell, and/or
(ii) TCR α and/or β chain sequences of a TCR expressed by the activated T cell.

2. The method of claim 1, wherein the composition sorted in (I) comprises a plurality of unique biological samples, and wherein the method comprises, before sorting, one or both of the following step(s):
creating a plurality of biological samples by equally distributing a collection of cells comprising T cells and antigen presenting cells (APCs) isolated from a subject into individual samples, wherein each biological sample optionally comprises media and cytokines that support T cell and/or APC viability, activation and/or activity, and
creating a plurality unique biological samples by delivering to each of a plurality of biological samples a unique antigen and/or a unique HTO that is used to specifically identify the unique antigen, wherein the unique HTO is conjugated to a molecule that labels a T cell with the unique HTO,
wherein each of the plurality of biological samples comprises a collection of cells comprising T cells and APCs isolated from a subject and optionally medium that supports viability, activity, and/or activation of the T cells and APCs, and
wherein after delivery of the unique antigen and/or the unique HTO conjugated to a molecule that labels a T cell with the unique HTO, each of the plurality of biological samples becomes a unique biological sample that comprises
(a) a collection of cells comprising T cells and APCs isolated from a subject,
(b) a unique antigen,
(c) a unique HTO that specifically identifies the unique antigen and is conjugated to a molecule that labels the T cell with the HTO, and optionally
(d) medium that supports viability, activity, and/or activation of the T cells and APCs.

3. The method of claim 2, wherein the APCs comprise monocyte-derived dendritic cells, dendritic cells, monocytes, macrophages, B cells, or a combination thereof.

4. The method of claim 1, wherein sorting comprises fluorescence activated cell sorting of activated T cells based on the expression of the AIM.

5. The method of claim 4, wherein the AIM is selected from the group consisting of CD137/4-1BB, CD107, IFNγ, PD-1, CD40L, OX40, CD25, CD69, CD28, HLA-DR, CX3CR1, TIM3, LAG3, TIGIT, and any combination thereof.

6. The method of claim 4, wherein fluorescence activated cell sorting is based on detection with fluorescently labeled antibody to the AIM.

7. The method of claim 1, comprising performing further functional and/or phenotypic analysis on the activated T cell analyzed in II, optionally wherein the further functional and/or phenotypic analysis is selected from the group consisting of flow cytometric analysis, CITE-seq analysis, multimer analysis, and a combination thereof.

8. The method of claim 7, wherein the further functional and/or phenotypic analysis measures the protein and/or RNA expression levels of one or more of CD3, CD4, CD8, CD25, CD27, CD28, CD45RA, CD62L, HLA-DR, CD137/4-1BB, CD69, CD278, CD274, CD279, CD127, CD197, IFNγ, GZMH, GNLY, CD38, CCL3, and LAG3.

9. The method of claim 1, wherein the T cell and surface bound MHC are provided by peripheral blood mononuclear cells.

10. The method of claim 1, wherein the molecule that labeled the T cell with the unique HTO comprises an antibody that binds a cell surface molecule.

11. The method of claim 1, wherein the AIM is or comprises CD137/4-1BB.

12. The method of claim 1, wherein the method comprises identifying a TCR α chain sequence and/or a TCR β chain sequence of a TCR that specifically binds the antigen, and
wherein the TCR α chain sequence and/or a TCR β chain sequence is or comprises a TCR α chain CDR3 sequence and/or a TCR β chain CDR3 sequence, respectively.

13. The method of claim 12, wherein the TCR α chain sequence and/or a TCR β chain sequence is a TCR α chain variable region sequence and/or a TCR β chain variable region sequence, respectively, and
wherein the method further comprises utilizing the TCR α chain variable region sequence and/or a TCR β chain variable region sequence in making a human therapeutic.

14. A composition comprising a biological sample that comprises:
(a) a T cell and a surface bound Major Histocompatibility Complex (MHC), wherein the T cell is capable of recognizing a peptide presented in the context of the surface bound MHC,
(b) an antigen,
(c) a hashtag oligonucleotide (HTO) that specifically identifies the antigen, wherein the HTO is conjugated to a molecule that labels the T cell with the HTO,
and optionally
(d) medium that supports activation of the T cell.

15. The composition of claim 14, wherein
(a) the MHC is expressed on the surface of an antigen presenting cell (APC), optionally
wherein:
the T cell and APC are autologous,
the T cell and the APC are each isolated from a human donor, and/or
the APC is selected from the group consisting of a monocyte-derived dendritic cell, a dendritic cell, a monocyte, a macrophage, a B cell and a combination thereof,
(b) the antigen
(I) is selected from the group consisting of
(i) a bacterial antigen or portion thereof,
(ii) a viral antigen or portion thereof,
(iii) an allergen or portion thereof,
(iv) a tumor associated antigen or a portion thereof, and
(v) a combination thereof, and/or
(II) comprises
(i) an amino acid sequence,
(ii) a nucleotide sequence,
(iii) lysate, and
(iv) a combination thereof,
(c) the HTO conjugated molecule comprises an antibody that binds a cell surface molecule or a lipid, and/or
(d) the medium comprises a cytokine that supports the viability of the T cell and/or an APC.

16. The composition of claim 15, wherein
the antibody binds a cell surface marker selected from the group consisting of β2 microglobulin, CD298, CD2, CD3, CD4, CD8, and any combination thereof, or
the lipid that incorporates into a cell membrane.

17. The composition of claim 15, wherein the cytokine that supports the viability of the T cell and/or the APC is selected from the group consisting of IL-2, IL-7, IL-15, GM-CSF, IL-4, and any combination thereof.

18. The composition of claim 15, further comprising a second biological sample, wherein the second biological sample comprises:
(a) a second T cell and a second surface bound MHC, wherein the second T cell is capable of recognizing a peptide presented in the context of the second surface bound MHC,
(b) a second antigen,
(c) a second HTO that specifically identifies the second antigen, wherein the HTO is conjugated to a second molecule that labels the second T cell with the second HTO,
and optionally
(d) a medium that supports activation of the second T cell,
wherein
(i) the T cell and second T cell are isolated from the same subject,
(ii) the antigen and the second antigen are not identical,
(iii) the molecule that labels the T cell with the HTO and the second molecule that labels the second T cell with the second HTO are identical, and the HTO and the second HTO are not identical.

19. The composition of claim 14, wherein the composition further comprises an agent that allows sorting an activated T cell based on expression of an activation-induced marker (AIM).

20. The composition of claim 19, wherein the agent that allows sorting activated T cell based on expression of an AIM is a fluorescently labeled antibody that specifically binds the AIM.

21. The composition of claim 19, wherein the AIM is selected from the group consisting of CD137/4-1BB, CD107, IFNγ, PD-1, CD40L, OX40, CD25, CD69, CD28, HLA-DR, CX3CR1, TIM3, LAG3, and/or TIGIT.

22. The composition of claim 14, wherein the composition comprises an antibody and/or MHC multimers useful for flow cytometric analysis or CITE-seq analysis of the composition.

23. A kit comprising
a plurality of unique antigens, and
a plurality of unique hashtag oligonucleotides (HTOs), each of which specifically identifies only one of the plurality of unique antigens.

24. The kit of claim 23, wherein the kit further comprises an agent that allows sorting of activated T cells based on their expression of an activation-induced marker (AIM), optionally wherein the agent that allows sorting activated T cell based on expression of an AIM is a fluorescently labeled antibody that specifically binds the AIM.

25. The kit of claim 23, wherein each of the plurality of unique HTOs is conjugated to an identical molecule such that the kit comprises a plurality of unique HTO-conjugated molecules.

26. The kit of claim 23, wherein each of the plurality of unique antigens comprises unique and overlapping peptide sequences from a single protein.

27. The kit of claim 26, wherein the single protein is selected from the group consisting of a pathogenic antigen, a tumor associated antigen, or a transplantation antigen.

28. The method of claim 1, further comprising analyzing a T cell mediated immune response of a patient:
(i) to a vaccine,
(ii) to an immunotherapy,
(iii) during immunotherapy of the patient,
(iv) to an autoantigen, or
(v) to a transplant antigen.

29-33. (canceled)

34. The method of claim 12, wherein the TCR α chain sequence and/or a TCR β chain sequence is a CDR3 sequence of a TCRα chain and/or a CDR3 sequence of a TCRβ chain, and
wherein the method further comprises utilizing the CDR3 sequence of a TCRα chain and/or a CDR3 sequence of a TCRβ chain in making a human therapeutic.

35.-36. (canceled)
